(19) United States
(12) Patent Application Publication
(10) Pub. No.: US 2026/0209143 A1
(43) Pub. Date: Jul. 23, 2026

(54) METHOD FOR MANUFACTURE OF STYLENE MONOMER

(71) Applicant: PS JAPAN CORPORATION, Tokyo (JP)

(72) Inventors: Michihiro ISHIHARA, Tokyo (JP); Sho TAMURA, Tokyo (JP)

(73) Assignee: PS JAPAN CORPORATION, Tokyo (JP)

(21) Appl. No.: 19/079,466

(22) Filed: Mar. 14, 2025

(30) Foreign Application Priority Data
Mar. 18, 2024 (JP) 2024-042869

(51) Int. Cl. C07C 4/22; C07C 7/04

(52) U.S. Cl. CPC C07C 4/22; C07C 7/04

(57) ABSTRACT

A method for producing a styrene monomer capable of suppressing the reduction in the production volume of styrene per unit time is provided. The present disclosure relates to a method for producing a styrene monomer, including a mixed liquid preparation step of preparing a mixed liquid by mixing a styrene-based resin composition containing a styrene-based polymer that includes styrene monomer units with a solvent to prepare a mixed liquid, a purification step of purifying the mixed liquid using a purifier; a devolatilization step of devolatilizing the purified mixed liquid to obtain a fluid, and a first thermal decomposition step of thermally decomposing the fluid to produce a first thermal decomposition liquid.

### TECHNICAL FIELD

The present disclosure relates to a method for producing a styrene monomer.

### BACKGROUND

In view of the recent trend of sustainable development goals (SDGs), technologies for recycling plastic materials such as polystyrene resins are in demand. Among these, as a method of recycling polystyrene resins, attention has been attracted to a method called mechanical recycling, which aims to reuse polystyrene resins without reverting them once to styrene monomer, and a method called chemical recycling, which reverts polystyrene resins once to styrene monomer.

With regard to the mechanical recycling of polystyrene resin, for example, the technique disclosed in PTL 1 is known. PTL 1 discloses a technique in which polystyrene resin waste is dissolved in a good solvent such as cymene, p-xylene, toluene, and ethylbenzene, insoluble substances are removed, and then the polystyrene resin component is precipitated and recovered by mixing with a poor solvent such as heptane.

Additionally, PTL 2 discloses a technique in which polystyrene waste is dissolved in limonene or an organic solvent with a boiling point of less than 200° C., insoluble substances are removed, and then vacuum heating devolatilization is performed to recover polystyrene resins without reducing the molecular weight of the polystyrene waste.

On the other hand, with regard to the chemical recycling of polystyrene resins, the technique disclosed in PTL 3 is known, for example. PTL 3 discloses a technique in which polystyrene resin waste is thermally melted, the thermally melted polystyrene resin is introduced into a tubular thermal decomposition apparatus and is further subjected to thermal decomposition at high heat to obtain styrene monomer.

Similarly, PTL 4 discloses a technique in which a polystyrene resin is thermally melted at high heat using a twin-screw extruder and further thermally decomposed to obtain styrene monomer.

### CITATION LIST

PTL 1: WO 2023/082009 A1
PTL 2: JP 2000-334738 A
PTL 3: JP 2001-40136 A
PTL 4: JP 2021-134281 A

### SUMMARY

The above-mentioned conventional techniques for the mechanical recycling of styrene resin involve dissolving polystyrene waste once using two or more organic solvents to remove foreign substances and the like, and then performing precipitation or devolatilization using a poor solvent to recover a high-quality recycled polystyrene resin. However, it has been demonstrated that, depending on the intended use, such as when the recycled polystyrene resin is used for food hygiene applications, the purity of the recycled polystyrene resin is not always sufficient. Therefore, the removal of impurities that meet various standards is currently required depending on the application.

Furthermore, all of the above-mentioned conventional techniques for the chemical recycling of styrene resins involve a step of thermally decomposing polystyrene waste, and a thermal decomposition step is hence currently substantially unavoidable when converting polymers into monomers through chemical recycling. Therefore, mechanisms for removing thermal decomposition residues or solid residues generated during thermal decomposition are disclosed in the above-mentioned PTLs 3 and 4.

However, polystyrene waste is generally not composed solely of pure polystyrene resin, and it may inevitably contain not only other resins such as polyolefin resins used in packaging and the like, for example, but also inorganic substances such as metal powder. Additionally, during the reduction process from polystyrene waste to styrene monomer through thermal decomposition, it has been demonstrated that the above-mentioned other resins and inorganic substances exhibit decomposition behavior different from that of polystyrene resins, and they do not merely become thermal decomposition residues.

Specifically, it has been demonstrated that low-fluidity liquids or solids are produced at temperatures lower than the thermal decomposition temperature in the gas generated by thermal decomposition, leading to new problems such as the blockage of the thermal decomposition apparatuses or piping that connects apparatuses, or the reduction in continuous operability of the thermal decomposition step, in addition to thermal decomposition residues containing carbides and inorganic substances generated during thermal decomposition.

Accordingly, the present disclosure is directed to suppress the reduction in the production volume of styrene per unit of average operating time during a long-term operation.

As a result of intensive studies to solve the above issues, the present inventor has discovered that by adding a solvent to dissolve a styrene-based resin composition containing a styrene-based polymer that includes styrene monomer units to obtain a mixed liquid, purifying and devolatilizing the mixed liquid to obtain a fluid, and then thermally decomposing the obtained fluid to obtain a first thermal decomposition liquid, it is possible to suppress the blockage of piping caused by residues generated in the thermal decomposition step or gases produced by thermal decomposition, as well as to suppress the reduction in the production volume of styrene of average operating time during a long-term operation, thereby completing the present disclosure.

Specifically, the present disclosure is as follows:

[1] A method for producing a styrene monomer, comprising:
a mixed liquid preparation step of preparing a mixed liquid by mixing a styrene-based resin composition containing a styrene-based polymer that includes styrene monomer units with a solvent;
a purification step of purifying the mixed liquid using a purifier;
a devolatilization step of devolatilizing the purified mixed liquid to obtain a fluid; and
a first thermal decomposition step of thermally decomposing the fluid to produce a first thermal decomposition liquid containing the styrene monomer.

[2] The method for producing a styrene monomer according to [1], further comprising a recovery step of recovering the styrene monomer by distilling the first thermal decomposition liquid.

[3] The method for producing a styrene monomer according to [1], wherein the solvent is one or more selected from the group consisting of toluene, methyl ethyl ketone, and ethylbenzene.

[4] The method for producing a styrene monomer according to any one of [1] to [3], wherein the fluid contains 10 mass % or more and 100 mass % or less of the styrene-based polymer per 100 mass % of a total amount of the fluid.

[5] The method for producing a styrene monomer according to any one of [1] to [4], wherein the purified mixed liquid contains 5 to 100 mass % of the styrene-based polymer per 100 mass % of a total amount of the purified mixed liquid.

[6] The method for producing a styrene monomer according to any one of [1] to [5], wherein the mixed liquid contains 5 mass % or more of the styrene-based polymer per 100 mass % of the total amount of the mixed liquid.

[7] The method for producing a styrene monomer according to any one of [1] to [6], wherein an ambient temperature for mixing the styrene-based resin composition with the solvent in the mixed liquid preparation step is 0° C. or higher.

[8] The method for producing a styrene monomer according to any one of [1] to [7], wherein the devolatilization step is performed under reduced pressure.

[9] The method for producing a styrene monomer according to any one of [1] to [8], wherein the first thermal decomposition step is performed under reduced pressure.

[10] The method for producing a styrene monomer according to any one of [1] to [9], further comprising an analysis step of analyzing the first thermal decomposition liquid.

[11] The method for producing a styrene monomer according to any one of [1] to [10], wherein the first thermal decomposition step comprises cooling a thermally decomposed vapor containing the styrene monomer to obtain the first thermal decomposition liquid.

[12] The method for producing a styrene monomer according to any one of [1] to [11], wherein the styrene-based resin composition contains 20 mass % or less of impurities.

[13] The method for producing a styrene monomer according to any one of [1] to [12], further comprising a second thermal decomposition step of distilling the first thermal decomposition liquid to separate the first thermal decomposition liquid into a first fraction containing the styrene monomer and a second fraction having a lower styrene monomer concentration than that in the first fraction, then distilling the first fraction to separate the first fraction into a third fraction having a higher styrene monomer concentration than that in the first fraction and a fourth fraction having a lower styrene monomer concentration than that in the first fraction, and then thermally decomposing the fourth fraction again to produce a second thermal decomposition liquid.

[14] The method for producing a styrene monomer according to [13], further comprising a recycling step of distilling the second thermal decomposition liquid to separate the second thermal decomposition liquid into a fifth fraction containing the styrene monomer and a sixth fraction having a lower styrene monomer concentration than that in the fifth fraction, and then recovering the styrene monomer from the fifth fraction.

[15] The method for producing a styrene monomer according to [14], wherein the recycling step further comprises a step (I) of recovering the styrene monomer using the fifth fraction as a part of the first thermal decomposition liquid, and a step (II) of recovering the styrene monomer by distilling the fifth fraction separately from the first thermal decomposition liquid.

[16] The method for producing a styrene monomer according to any one of [1] to [15], wherein the first thermal decomposition step comprises heating the fluid to obtain the thermally decomposed vapor containing the styrene monomer and then cooling the thermally decomposed vapor to produce the first thermal decomposition liquid, wherein a concentration of a deposit-inducing substance contained in the first thermal decomposition liquid is less than 0.2 mass %.

[17] The method for producing a styrene monomer according to any one of [1] to [16], wherein the devolatilization step is a step of devolatilizing the mixed liquid to obtain the fluid using a flash drum, flash tank polymer heater, twin-screw devolatilizer, thin-film evaporator, or extruder.

[18] The method for producing a styrene monomer according to any one of [1] to [17], wherein the purifier employs one or more selected from the group consisting of filtration, decantation, centrifugal separation, centrifugal sedimentation, screw decanter, strainer, screen mesh, and filter.

[19] The method for producing a styrene monomer according to any one of [1] to [18], wherein the purifier is a purification mechanism combining centrifugal separation and filtration.

According to the present disclosure, it is possible to provide a method for suppressing the reduction in the production volume of styrene per unit of average operating time during a long-term operation.

According to the present disclosure, it is possible to provide a method that contributes to the extension of continuous operation time.

According to the present disclosure, it is possible to provide a method with which by-products in the thermal decomposition liquid obtained in the thermal decomposition steps are reduced, the load on the subsequent distillation step is reduced, and continuous operation is achieved without the need for cumbersome maintenance.

### BRIEF DESCRIPTION OF THE DRAWINGS

In the accompanying drawings:

FIG. 1 is a flow diagram illustrating one example of a method for producing a styrene monomer from a styrene-based resin composition according to the present embodiment;

FIG. 2 is a flow diagram illustrating another example of a method for producing a styrene monomer from a styrene-based resin composition according to the present embodiment;

FIG. 3 is a schematic diagram illustrating one example of a thermal decomposition apparatus used in the method for producing a styrene monomer from a styrene-based resin composition according to the present embodiment;

FIG. 4 is a flow diagram illustrating another example of a method for producing a styrene monomer from a styrene-based resin composition according to the present embodiment;

FIG. 5 is a schematic diagram illustrating one example of a production apparatus for styrene monomer according to the present embodiment;

FIG. 6 is a photograph indicating the connecting section C1P, which is a glass tube immediately before the production of styrene monomer, in a case where styrene monomer is produced using an apparatus in which the thermal decomposition apparatus 10 and a liquefaction apparatus L illustrated in FIG. 3 are connected via the connecting section C1P; more specifically, FIG. 6 is a photograph indicating the connecting section C1P, which is a glass tube immediately before the production of styrene monomer in Comparative Example 5;

FIG. 7 is a photograph indicating the connecting section C1P, which is a glass tube during the production of styrene monomer in Comparative Example 5, where the styrene monomer is produced using an apparatus in which the thermal decomposition apparatus 10 and the liquefaction apparatus L illustrated in FIG. 3 are connected via the connecting section C1P; and

FIG. 8 is a photograph indicating the connecting section C1P, which is a glass tube immediately after the production of styrene monomer in Comparative Example 5, where the styrene monomer is produced using an apparatus in which the thermal decomposition apparatus 10 and the liquefaction apparatus L illustrated in FIG. 3 are connected via the connecting section C1P.

### DETAILED DESCRIPTION

The following provides a detailed description of an embodiment of the present disclosure (hereinafter, referred to as “present embodiment”). However, the present disclosure is not limited by the description given below, and may be implemented with various changes or modifications that are within the essential scope thereof.

### [Method for Producing a Styrene Monomer]

The method for producing a styrene monomer of the present disclosure is a method for thermally decomposing a styrene-based resin composition containing a styrene-based polymer that includes styrene monomer units. The styrene-based polymer may be a resin containing styrene monomer units that has been used, discarded, or is to be discarded. In other words, the method for producing a styrene monomer of the present embodiment comprises a mixed liquid preparation step of preparing a mixed liquid by mixing a styrene-based resin composition with a solvent, a purification step of purifying the mixed liquid using a purifier, a devolatilization step of devolatilizing the purified mixed liquid to obtain a fluid, and a first thermal decomposition step of thermally decomposing the fluid to produce a first thermal decomposition liquid containing the styrene monomer.

This method makes it possible to suppress not only the thermal decomposition residue containing carbides and inorganic substances generated during thermal decomposition but also factors that reduce the continuous operability of the thermal decomposition step, such as the blockage of the thermal decomposition apparatus and piping connecting apparatuses.

In addition, it is preferable that the method for producing a styrene monomer further comprises a recovery step of recovering the styrene monomer by distilling the first thermal decomposition liquid. This enables the production of a high-purity styrene monomer.

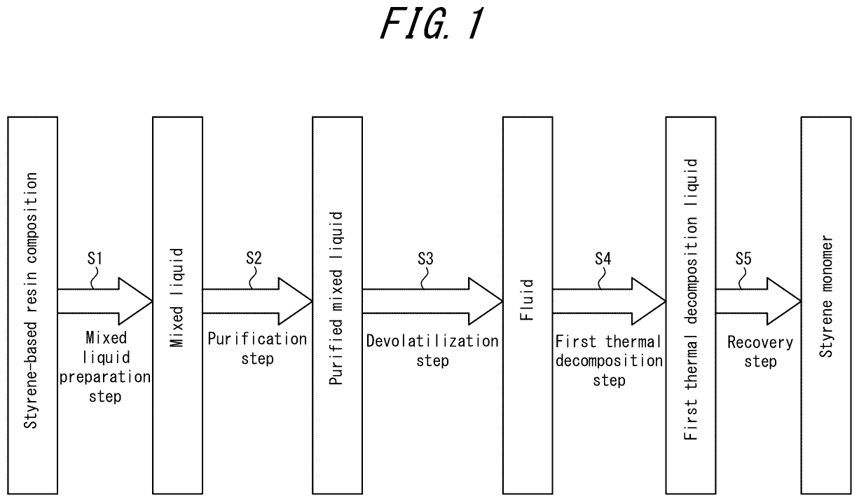

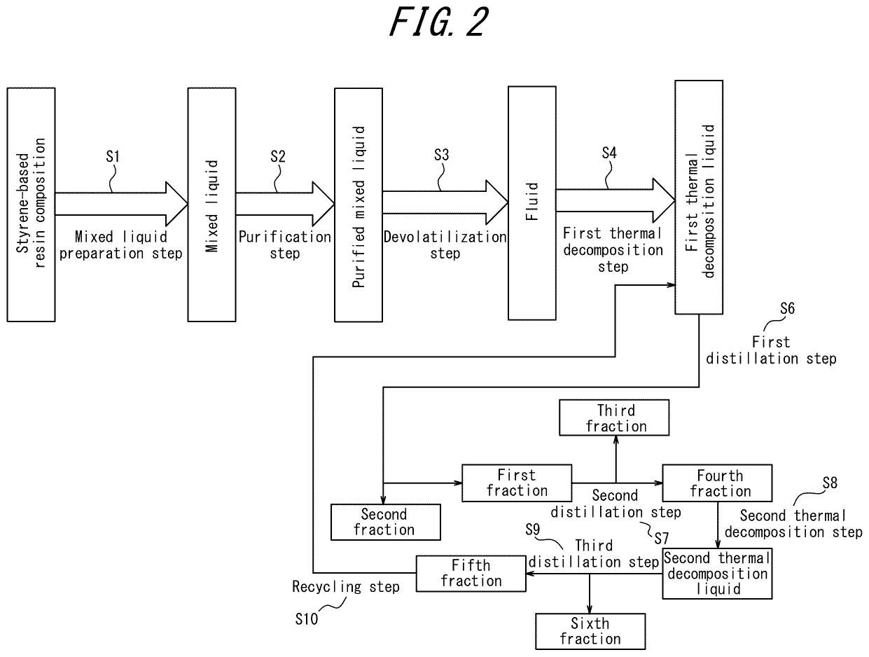

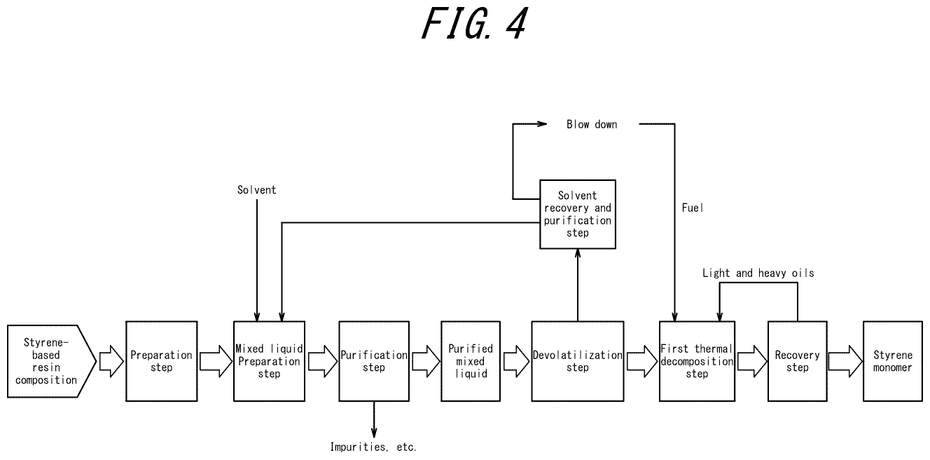

Hereinafter, the summary of the method for producing a styrene monomer of the present embodiment will be described with reference to FIG. 1 and FIG. 2, followed by a detailed description of each of steps (S1) to (S10). Additionally, as one example of a preferred embodiment of the method for producing a styrene monomer of the present embodiment, a flow diagram is illustrated in FIG. 4.

FIG. 1 is a flow diagram illustrating one example of a method for producing a styrene monomer from a styrene-based resin composition according to the present embodiment. More specifically, FIG. 1 illustrates a mixed liquid preparation step (S1) of preparing a mixed liquid by mixing a styrene-based resin composition as the raw material with a solvent, a purification step (S2) of purifying the mixed liquid to concentrate the styrene-based polymer contained in the mixed liquid, a devolatilization step (S3) of devolatilizing the purified mixed liquid to prepare a fluid from which a certain amount of the solvent has been distilled off, a first thermal decomposition step (S4) of thermally decomposing the fluid to produce a first thermal decomposition liquid containing the styrene monomer, and an optional recovery step (S5) of recovering the styrene monomer from the first thermal decomposition liquid.

FIG. 2 is a flow diagram illustrating another example of a method for producing a styrene monomer from a styrene-based resin composition according to the present embodiment.

More specifically, FIG. 2 illustrates a mixed liquid preparation step (S1) of preparing a mixed liquid by mixing a styrene-based resin composition as the raw material with a solvent, a purification step (S2) of purifying the mixed liquid to concentrate the styrene-based polymer contained in the mixed liquid, a devolatilization step (S3) of devolatilizing the purified mixed liquid to prepare a fluid from which a certain amount of the solvent has been distilled off, a first thermal decomposition step (S4) of thermally decomposing the fluid to produce a first thermal decomposition liquid containing the styrene monomer, an optional first distillation step (S6) of distilling the first thermal decomposition liquid to separate the first thermal decomposition liquid into a first fraction containing the styrene monomer and a second fraction having a lower styrene monomer concentration than that in the first fraction, an optional second distillation step (S7) of distilling the first fraction to further separate the first fraction into a third fraction containing the styrene monomer and a fourth fraction having a lower styrene monomer concentration than that in the third fraction, an optional second thermal decomposition step (S8) of thermally decomposing the fourth fraction again to produce a second thermal decomposition liquid, an optional third distillation step (S9) of distilling the second thermal decomposition liquid to separate the second thermal decomposition liquid into a fifth fraction containing the styrene monomer and a sixth fraction having a lower styrene monomer concentration than that in the fifth fraction, an optional recycling step (S10) of recovering the styrene monomer from the fifth fraction, and an optional recovery step (S5) of recovering the styrene monomer from the first thermal decomposition liquid.

FIG. 4 is a flow diagram illustrating another example of a method for producing a styrene monomer from a styrene-based resin composition according to the present embodiment. More specifically, FIG. 4 illustrates a preparation step of preparing a styrene-based resin composition as the raw material, a mixed liquid preparation step (S1) of preparing a mixed liquid by blending a solvent with the styrene-based resin composition as the raw material, a purification step (S2) of purifying the mixed liquid to concentrate the styrene-based polymer contained in the mixed liquid and remove impurities and the like contained in the styrene-based resin composition as the raw material, a devolatilization step (S3) of devolatilizing the purified mixed liquid to prepare a fluid from which a certain amount of the solvent has been distilled off, a first thermal decomposition step (S4) of thermally decomposing the fluid to produce a first thermal decomposition liquid containing the styrene monomer, and an optional recovery step (S5) of recovering the styrene monomer from the first thermal decomposition liquid. Additionally, the devolatilization step (S3) may optionally comprise a step (i.e., solvent recovery and purification step) of recovering the solvent distilled from the purified mixed liquid and purifying the solvent by distillation. The recycled solvent purified by the solvent recovery and purification step may be reused in the mixed liquid preparation step (S1). Additionally, the residue other than the recycled solvent obtained from the solvent recovery and purification step may be blown down and used as fuel for the first thermal decomposition step (S4).

Additionally, components other than the styrene monomer obtained in the recovery step (S5), i.e., light fractions or heavy fractions (light and heavy oils), may be used as fuel for the first thermal decomposition step (S4). As a result, an improvement in the yield of the obtained styrene monomer can be expected.

Each of the steps will be described below.

### (Mixed Solution Preparation Step: S1)

The method for producing a styrene monomer of the present embodiment comprises a step of preparing a mixed liquid by mixing a styrene-based resin composition with a solvent. This step achieves the effect of concentrating the styrene-based polymer utilizing the solubility of the solvent.

### <Solvent>

The solvent of the present embodiment is preferably capable of dissolving the styrene-based polymer in the styrene-based resin composition. This allows separation into components containing the styrene-based polymer that dissolves in the solvent and components that are insoluble or poorly soluble in the solvent. As a result, the styrene-based polymer can be concentrated.

In the present embodiment, it is preferable that the solubility parameter (SP value ((cal/cm³)^{1/2}) of the solvent is 8.0 or more and less than 11.0, more preferably 8.3 or more and less than 10.5, and even more preferably 8.6 or more and less than 10.0. When the solubility parameter of the solvent is within the above range, it becomes easier to selectively dissolve styrene-based polymers (for example, polystyrene, styrene-(meth)acrylic acid-based copolymers, etc.).

Preferred solvents in the present embodiment include the following solvents. The solvent is preferably an organic solvent, and specifically, the organic solvent may be one or more solvents selected from the group consisting of acetone (9.9), chloroform (9.3), methyl ethyl ketone (9.3), benzene (9.2), tetrahydrofuran (9.1), toluene (8.9), ethylbenzene (8.8), and styrene (9.3). The values in parentheses indicate the corresponding SP values of the solvents. The solvent may be used alone or as a mixed solvent containing two or more solvents.

The solubility parameter (SP value) defined in the present is calculated using the function of cohesive energy density expressed by the following formula (1):

$$SP value ( {( cal / {cm}^{3} )}^{1 / 2} ) = {( Δ E / {V}^{3} )}^{1 / 2} \qquad ( 1 )$$

in the formula (1), ΔE represents the intermolecular cohesive energy (evaporation heat), V represents the total volume of the liquid mixture, and ΔE/V represents the cohesive energy density.
The heat quantity change ΔHm due to mixing is expressed by the following formula (2) using SP values:

$$Δ Hm = V ( δ1 - δ2 ) \cdot Φ1 \cdot Φ2 \qquad ( 2 )$$

in the formula (2), δ1 represents the SP value of the solvent, δ2 represents the SP value of the solute, Φ1 represents the volume fraction of the solvent, and Φ2 represents the volume fraction of the solute.)
From the above formulas (1) and (2), when δ1 and δ2 are closer, ΔHm is smaller, and the Gibbs free energy is smaller. Thus, the affinity is higher when the difference in SP value is smaller.

The above SP values are each SP values obtained using the Hildebrand method (including the Hansen method) and the values described in the literature (J. Brandrup, E. H. Immergut, E. A. Grulke, Polymer Handbook 4^{th} Edition, WILEY-INTERSCIENCE) were used.

The solvent in the present embodiment is preferably an organic solvent, and may be a mixed solvent containing two or more organic solvents.

Moreover, it is more preferable that the number of carbon atoms of each component constituting the organic solvent is 8 or less, and even more preferably 1 or more and 8 or less.

By setting the number of carbon atoms in the organic solvent to 8 or less, it is possible to ensure the solubility of the styrene-based polymer while suppressing an increase in the boiling point of the solvent per se, and the amount of solvent remaining in the fluid can be kept low in the devolatilization step of devolatilizing the purified mixed liquid containing the styrene-based polymer to obtain a fluid.

Furthermore, by using an organic solvent that has 1 or more and 8 or fewer carbon atoms and has a solubility parameter (SP value ((cal/cm³)^{1/2}) of 8.0 or more and less than 11.0, the solubility differences of each component in the organic solvent can be utilized to promote the selective separation of impurities and/or deposit-inducing substances from the styrene-based polymer and/or styrene monomer.

The content of the solvent in the present embodiment is preferably 5 to 95 mass %, more preferably 35 to 90 mass %, and even more preferably 50 to 80 mass % relative to the entire mixed liquid.

When the amount of the solvent mixed is within the above range, it becomes easier to separate the component containing the styrene-based polymer dissolved in the solvent from other components.

### <Styrene-Based Resin Composition>

The styrene-based resin composition used in the method for producing a styrene monomer of the present embodiment only needs to contain a styrene-based polymer that includes styrene monomer units. The lower limit of the content of the styrene-based polymer contained in the styrene-based resin composition used as the raw material in the method for producing a styrene monomer is preferably 50 mass % or more, more preferably 60 mass % or more, even more preferably 70 mass % or more, and still even more preferably 80 mass % or more relative to the entire styrene-based resin composition.

On the other hand, the upper limit of the content of the styrene-based polymer may be 50 mass %, 60 mass %, 70 mass %, 80 mass %, or 100 mass % or less relative to the entire styrene-based resin composition.

When the content of the styrene-based polymer contained in the styrene-based resin composition is within the above range, the yield of the thermal decomposition liquid produced in the thermal decomposition step described later is increased, resulting in an increased yield of the styrene monomer in the recovery step.

The styrene-based resin composition in the present embodiment includes used, discarded, or waste materials, pre-consumer materials such as factory-recovered products, post-consumer materials such as market-recovered products, long-term stock pellets, and off-specification pellets. Additionally, the styrene-based resin composition may contain additives such as phosphorus-based flame retardants, liquid paraffin, stabilizers, or colorants.

The styrene-based resin composition may also contain impurities. It is preferable that the styrene-based resin composition in the present embodiment is a used, discarded, or waste material, in other words, it is preferable that the styrene-based resin composition is a recycled polystyrene-based resin composition. When the styrene-based resin composition in the present embodiment is a recycled polystyrene-based resin composition, it may contain impurities as contaminants derived from recycling. Such impurities may include, for example, resins other than styrene-based polymers (e.g., olefin resins such as polyethylene-based resins and polypropylene-based resins), inorganic substances (e.g., metal powder or silica), pigments, dyes, fine particles, adhered substances, or foreign matter.

More specifically, the term “impurities” in this specification may include other resins that substantially do not contain styrene monomer units, such as olefinic resins, polyether-based resins, polyester-based resins, or polyamide-based resins, for example. Furthermore, the styrene-based resin composition may be a laminated product with these other resins, or may be a mixed resin in which other resins such as olefinic resins, polyether-based resins, polyester-based resins, and polyamide-based resins are mixed with a styrene-based polymer containing styrene monomer units. In another aspect of the styrene-based resin composition in the present embodiment, the styrene-based resin composition may be a composition in which cellulose-based materials represented by paper (such as paper labels), thermosetting resins represented by phenolic resins, polyurethane-based resins, epoxy resins, and melamine-based resins, and a styrene-based polymer containing styrene monomer units are mixed. Furthermore, the styrene-based resin composition may contain fillers such as silicate minerals represented by talc, inorganic substances such as glass, and fibers used in fiber-reinforced plastics, such as carbon fibers, glass fibers, and cellulose fibers. Additionally, metals such as aluminum, iron, and SUS, may be contained.

The upper limit of the amount of impurities contained in the styrene-based resin composition used as the raw material in the method for producing a styrene monomer is preferably 50 mass % or less, more preferably mass % or less, even more preferably 30 mass % or less, and still even more preferably 20 mass % or less relative to the entire styrene-based resin composition. On the other hand, the lower limit of the amount of impurities contained in the styrene-based resin composition is preferably 0 mass % or more, and more preferably more than 0 mass % relative to the entire styrene-based resin composition.

In particular, it is beneficial that post-consumer materials, which gives thermal decomposition residues during thermal decomposition, are used for the method for producing a styrene monomer of the present embodiment. However, even if the styrene-based resin composition in the present embodiment is a virgin polystyrene-based resin composition containing an unused styrene-based polymer or a pre-consumer material such as factory-recovered products, it may contain the above-mentioned additives, or other resins or inorganic substances unintentionally mixed in during processing. Therefore, the method for producing a styrene monomer of the present embodiment is advantageous as a method capable of producing a styrene monomer while suppressing thermal decomposition residues from these virgin polystyrene-based resin compositions or pre-consumer materials.

### <<Styrene-Based Polymer>>

The styrene-based resin composition usable in the present embodiment contains a styrene-based polymer that includes styrene monomer units. The styrene-based resin composition or the styrene-based polymer may be a used, discarded, or waste material. The styrene-based polymer only needs to have styrene monomer units and is preferably a polymer obtained by polymerizing styrene monomer units with at least one other component selected from vinyl-based monomer units capable of copolymerizing with the styrene monomer and rubbery polymers, as needed. In other words, it is preferable that the styrene-based polymer contained in the styrene-based resin composition has styrene monomer units, and it is more preferable that the styrene-based polymer essentially contains styrene monomer units and optionally includes vinyl-based monomers capable of copolymerizing with the styrene monomer units and/or rubbery polymer monomer units. The preferred forms of the styrene-based polymer in the present embodiment are not particularly limited. Specifically, examples include rubber-modified styrene-based resins in which rubbery polymer particles are dispersed in a polymer matrix containing polystyrene or polystyrene-based polymers (such as polystyrene and/or polystyrene-unsaturated carboxylic acid-based polymers), or styrene-based copolymer resins.

In the styrene-based polymer contained in the styrene-based resin composition usable in the present embodiment, the lower limit of the content of styrene monomer units is preferably 50 mass % or more, more preferably 60 mass % or more, and even more preferably 70 mass % or more, relative to the entire styrene-based polymer (100 mass %). On the other hand, the upper limit of the content of styrene monomer units may be 50 mass %, 60 mass %, 70 mass %, 80 mass %, or 100 mass %.

### —Polystyrene—

In the present embodiment, polystyrene refers to a polymer containing styrene monomer units and optionally other styrene-based monomer units. The monomers constituting polystyrene include styrene and, as optional components, other styrene-based monomers. Examples of other styrene-based monomers include α-methylstyrene, α-methyl-p-methylstyrene, o-methylstyrene, m-methylstyrene, p-methylstyrene, vinyltoluene, ethylstyrene, isobutylstyrene, and t-butylstyrene, or styrene derivatives such as bromostyrene and indene. One or more of these monomers may be used. The inclusion of monomer units other than those mentioned above in polystyrene is not excluded as long as the effects of the present disclosure are not impaired, but it is typically composed of styrene monomer units.

### —Rubber-Modified Styrene-Based Resin—

In the present embodiment, a rubber-modified styrene-based resin refers to a material in which particles of a rubbery polymer are dispersed in a matrix phase of a styrene-based polymer (e.g., polystyrene), and which has a sea-island structure where the matrix phase serves as the sea phase and the particles of the rubbery polymer (i.e., rubbery polymer particles) serve as the island phase. The rubber-modified styrene-based resin can be produced by polymerizing a styrene monomer (and other styrene-based monomers or unsaturated carboxylic acid-based monomers that are added if necessary) in the presence of a rubbery polymer.

It should be noted that the unsaturated carboxylic acid-based monomers include (meth)acrylic acid monomers and (meth)acrylic acid ester monomers.

The styrene monomer and other styrene-based monomers that are added if necessary constituting the rubber-modified styrene-based resin in the present embodiment are the same as those for the styrene monomer of polystyrene described above, and description thereof is thus omitted here.

The rubbery polymer particles contained in the rubber-modified styrene-based resin in the present embodiment may, for example, enclose a resin containing styrene monomer units derived from the above styrene monomer therein, and/or have a resin containing styrene monomer units grafted onto the surface thereof. More specifically, the rubbery polymer particles may contain polystyrene and/or a polystyrene-unsaturated carboxylic acid-based polymer, etc., enclosed therein. Similarly, polystyrene and/or a polystyrene-unsaturated carboxylic acid-based polymer may be grafted onto the surface of the rubbery polymer particles.

Examples of the rubbery polymer include polybutadiene, polyisoprene, natural rubber, polychloroprene, styrene-butadiene copolymer, and acrylonitrile-butadiene copolymer. Among these, polybutadiene or styrene-butadiene copolymer is preferred. Both high cis polybutadiene having a high cis content and low cis polybutadiene having a low cis content can be used as the polybutadiene. In addition, as the structure of the styrene-butadiene copolymer, both a random structure and a block structure can be used. One or more of these rubbery polymers may be used. A saturated rubber obtained by hydrogenating a butadiene-based rubber may be used.

Examples of such a rubber-modified styrene-based resin include a high impact polystyrene (HIPS), an acrylonitrile-butadiene-styrene copolymer (ABS resin), an acrylonitrile-acrylic rubber-styrene copolymer (AAS resin), and an acrylonitrile-ethylene propylene rubber-styrene copolymer (AES resin).

In the case where the rubber-modified styrene-based resin is an HIPS-based resin, particularly preferred among these rubbery polymer particles is a high cis polybutadiene including 90 mol % or more of cis-1,4 bonds. In the high cis polybutadiene, the vinyl 1,2 bond is included preferably in an amount of 6 mol % or less, particularly preferably 3 mol % or less.

It should be noted that the content ratio of the constitutional units of the above-described high cis polybutadiene having cis-1,4 structure, trans-1,4 structure, or vinyl-1,2 structure as isomers of the constitutional units of the high cis polybutadiene can be calculated by carrying out a measurement using an infrared spectrophotometer and data processing by the Morello method.

In addition, the above-described high cis polybutadiene can be easily obtained through polymerization of 1,3-butadiene using a well-known production method, for example, by using a catalyst containing an organoaluminum compound and a cobalt or nickel compound.

The content of the rubbery polymer contained in the rubber-modified styrene-based resin is preferably 3 to 20 mass %, and more preferably 5 to 15 mass % relative to the total amount of 100 mass % of the rubber-modified styrene-based resin.

It should be noted that the content of the rubbery polymer contained in the rubber-modified styrene-based resin in the present disclosure is a value calculated using pyrolysis gas chromatography.

The average particle diameter of the rubbery polymer particles contained in the rubber-modified styrene-based resin is preferably 0.5 to 4.0 μm, and more preferably 0.8 to 3.5 μm.

In the present disclosure, the average particle diameter of the rubbery polymer particles contained in the rubber-modified styrene-based resin is measured by the following method.

An ultrathin section with a thickness of 75 nm is prepared from the rubber-modified styrene-based resin stained with osmium tetroxide, and a photograph is taken at a magnification of 10,000× under an electron microscope. In the photograph, the darkly stained particles are the rubbery polymer (a). From the photograph, the area average particle diameter is calculated using the formula (N1) below, and this value is taken as the average particle diameter of the rubbery polymer particles:

$$Average diameter = \sum {niDri}^{3} / \sum {niDri}^{2} \qquad ( N1 )$$

in the above mathematical formula (N1), ni is the number of rubbery polymer particles having a particle diameter Dri, and Dri is the particle size calculated as the equivalent circle diameter from the area of the particles in the photograph.

This measurement is performed by scanning the photograph at a resolution of 200 dpi and analyzing the image using the particle analysis software of the image analysis apparatus IP-1000 (manufactured by Asahi Kasei Corporation).

The reduced viscosity of the rubber-modified styrene-based resin, which is an indicator of the molecular weight of the rubber-modified styrene-based resin, is preferably in a range of 0.50 to 0.85 dL/g, and more preferably in a range of 0.55 to 0.80 dL/g.

It should be noted that the reduced viscosity of the rubber-modified styrene-based resin in the present disclosure is a value measured under conditions of 30° C. and a concentration of 0.5 g/dL in a toluene solution.

### —Styrene-Based Copolymer Resin—

In the present embodiment, a styrene-based copolymer resin refers to a resin containing styrene monomer units and unsaturated carboxylic acid-based monomer units capable of copolymerizing with the styrene monomer. In the styrene-based copolymer resin according to the present embodiment, the content of styrene monomer units is preferably 69 to 98 mass %, more preferably 74 to 96 mass %, and even more preferably 77 to 92 mass %, when the total content of styrene monomer units and unsaturated carboxylic acid-based monomer units is taken as 100 mass %.

It should be noted that the unsaturated carboxylic acid-based monomers in the present embodiment include unsaturated carboxylic acid monomers (e.g., (meth)acrylic acid monomers) and unsaturated carboxylic acid ester monomers (e.g., (meth)acrylic acid ester monomers).

In the styrene-based copolymer resin in the present embodiment, when the total content of styrene monomer units, unsaturated carboxylic acid monomer units, and unsaturated carboxylic acid ester monomer units is taken as 100 mass %, the content of unsaturated carboxylic acid monomer units is preferably 2 to 16 mass %, more preferably 4 to 14 mass %, and even more preferably 8 to 13 mass %.

In the present embodiment, when the total content of styrene monomer units, unsaturated carboxylic acid monomer units, and unsaturated carboxylic acid ester monomer units is taken as 100 mass %, the content of unsaturated carboxylic acid ester monomer units is preferably 0 to 15 mass %, more preferably 1 to 12 mass %, and even more preferably 2 to 10 mass %.

In the present embodiment, the content of styrene monomer units, unsaturated carboxylic acid monomer units (e.g., methacrylic acid monomer units), and unsaturated carboxylic acid ester monomer units (e.g., methyl methacrylate monomer units) in the styrene-based copolymer resin can be determined from the integral ratio in a spectrum measured using a proton nuclear magnetic resonance (¹H-NMR) spectrometer.

In the present embodiment, the inclusion of monomer units other than styrene monomer units and unsaturated carboxylic acid-based monomers (e.g., unsaturated carboxylic acid monomer units and unsaturated carboxylic acid ester monomer units) in the styrene-based copolymer resin is not excluded as long as the effects of the present disclosure are not impaired. However, typically, the styrene-based copolymer resin in the present disclosure is preferably composed of styrene monomer units, unsaturated carboxylic acid monomer units, and/or unsaturated carboxylic acid ester monomer units.

The styrene-based copolymer resin of the present embodiment may contain other styrene-based monomers in addition to styrene monomer units.

The unsaturated carboxylic acid monomers constituting the styrene-based copolymer resin in the present embodiment are not particularly limited, but examples include (meth)acrylic acid (methacrylic acid and/or acrylic acid), maleic anhydride, maleic acid, fumaric acid, and itaconic acid.

The unsaturated carboxylic acid ester-based monomers constituting the styrenic copolymer resin in the present embodiment are not particularly limited, but examples include (meth)acrylic acid esters (e.g., methyl (meth)acrylate, ethyl (meth)acrylate, propyl (meth)acrylate, butyl (meth)acrylate, and cyclohexyl (meth)acrylate). These unsaturated carboxylic acid ester-based monomers may be used singly or in combination of two or more.

Preferred styrene-based copolymer resins in the present embodiment include styrene-methacrylic acid copolymer, styrene-methyl methacrylate copolymer, styrene-methacrylic acid-methyl methacrylate copolymer, styrene-acrylic acid copolymer, styrene-methyl acrylate copolymer, styrene-acrylic acid-methyl acrylate copolymer, styrene-methyl methacrylate-butyl methacrylate copolymer, styrene-butyl methacrylate copolymer, or styrene-maleic anhydride copolymer.

In the present embodiment, the weight average molecular weight (Mw) of the styrene-based copolymer resin is preferably 100,000 to 350,000, more preferably 120,000 to 300,000, and further preferably 140,000 to 240,000. The weight average molecular weight (Mw) is a value in terms of standard polystyrene obtained using gel permeation chromatography.

In the present embodiment, the polymerization method for the styrene-based polymer resin is not limited, and for example, a bulk polymerization method or a solution polymerization method can be suitably employed as a radical polymerization method. The polymerization method primarily includes a polymerization step of polymerizing polymerization raw materials (monomer components) and a devolatilization step of removing volatile components such as unreacted monomers and polymerization solvents from the polymerization product.

### —Optional Added Component—

The styrene-based resin composition in the present embodiment, in addition to the styrene-based polymer and impurities that are contained if necessary, may include optional additive components such as conventionally known additives and processing aids, as long as the effects of the present disclosure are not impaired. Examples of such additives, processing aids, and the like include antioxidants, weathering agents, lubricants, antistatic agents, and fillers.

Examples of the antioxidants include phenolic compounds, phosphorous compounds, and thioether compounds.

As the weathering agent, ultraviolet absorbers and the like can be used.

As the lubricant, fatty acid amides, fatty acid esters, fatty acids, and metal salts of fatty acids can be used.

As the antistatic agent, fatty acid partial esters such as cationic, anionic, nonionic, amphoteric, and glycerin fatty acid monoesters can be used.

As the filler, talc, calcium carbonate, barium sulfate, carbon fibers, glass fibers, cellulose fibers, mica, wollastonite, and whiskers can be used.

The styrene-based resin composition in the present embodiment may contain optional additive components such as the above-mentioned additives and processing aids, as well as blocking inhibitors, colorants, blooming inhibitors, surface treatment agents, antibacterial agents, and die-drool prevention agents (such as the die-drool prevention agent, e.g., a monoester compound produced by reacting silicone oil, a monoamide compound of a higher aliphatic carboxylic acid, and a higher aliphatic carboxylic acid with a univalent to trivalent alcoholic compound disclosed in JP 2009-120717 A). The total content of optional additive components such as additives and processing aids in the styrene-based resin composition is preferably 0 mass % to 6 mass %, more preferably 0.05 to 5 mass %.

### <Preferred Forms of Styrene-Based Resin Composition>

A preferred form of the styrene-based resin composition used as the raw material in the method for producing a styrene monomer of the present embodiment is a recycle styrene-based resin composition, wherein the total content of the styrene-based polymer and optional additive components is 50 to 100 mass % and the content of impurities is 1 to 30 mass % relative to the entire styrene-based resin composition, and the styrene-based resin composition is a styrene-based resin composition containing a styrene-based polymer to be recycled that has been used, discarded, or recycled after disposal.

By using a recycle styrene-based resin composition containing a styrene-based polymer to be recycled that has been used, discarded, or recycled after disposal as the raw material in the production method, the environmental impact can be reduced.

### <Preferred Forms of Mixed Solution>

A preferred form of the mixed liquid obtained through the mixed liquid preparation step in the present embodiment is a mixed liquid in which the total content of the styrene-based polymer, solvent, and optional additive components is preferably 50 to 100 mass %, more preferably 50 mass % or more and less than 100 mass %, and the content of impurities is preferably 0 to mass %, more preferably more than 0 mass % and 10 mass % or less, relative to the entire mixed liquid.

### <Mixing or Kneading Conditions in Mixed Solution Preparation Step>

The mixed liquid in the present embodiment only needs to contain a styrene-based resin composition containing a styrene-based polymer that includes styrene monomer units and a solvent. The dispersion state of the solvent in the styrene-based resin composition is not particularly limited, and pre-mixing may be performed before or during the purification step described later using a known mixing or kneading mechanism.

The mixing or kneading mechanism used to mix the styrene-based resin composition and the solvent is not particularly limited, but a method that ensures uniform mixing of each component in the mixed liquid is preferred. If necessary, melt mixing or melt kneading may also be used. Specific examples of the mixing or kneading mechanisms include a magnetic stirrer, Three-One Motor, single-screw extruder, twin-screw extruder, screw-type extruder, open roll, kneader, Banbury mixer, or internal mixer.

The mixing or kneading time in the mixed liquid preparation step is preferably about 1 to 240 minutes, more preferably 1 to 120 minutes, even more preferably 2 to 120 minutes, and still even more preferably 5 to 120 minutes.

The temperature of the mixing atmosphere in the mixed liquid preparation step is preferably 0 to 260° C., more preferably 10 to 100° C., and even more preferably 20 to 80° C. Furthermore, when mixing, it is preferable that the pressure difference between atmospheric pressure and the pressure inside the mixing mechanism (or mixing apparatus) is within ±500 hPa, more preferably within ±300 hPa. If necessary, the inside of the mixing mechanism (or mixing apparatus) may be replaced with an inert gas. Specific examples of the inert gas include nitrogen gas, argon gas, and carbon dioxide gas.

### <Preparation Step>

The method for producing a styrene monomer in the present disclosure may include, for example, a preparation step in which the styrene-based resin composition as the raw material is prepared before the mixed liquid preparation step. The preparation step comprises, for example, a step of collecting and recycling styrene-based resin-containing waste plastic (post-consumer products) from the market or consumers, as the styrene-based resin composition used as the raw material (post-consumer step), and/or a step of collecting and recycling unused products such as scraps generated during manufacturing, before being distributed to consumers or the market (pre-consumer products), as the styrene-based resin composition used as the raw material (pre-consumer step).

The preparation step of preparing the styrene-based resin composition from post-consumer and/or pre-consumer products preferably comprises a crushing step of crushing styrene-based resin-containing waste plastic, a washing step of washing the crushed waste plastic, or a sorting step of sorting the crushed waste plastic.

On the other hand, the preparation step of preparing the styrene-based resin composition from pre-consumer materials such as virgin pellets preferably comprises a crushing step of crushing virgin materials containing styrene-based resin.

### (Purification Step: S2)

The method for producing a styrene monomer of the present embodiment comprises a purification step of purifying the mixed liquid prepared in the mixed liquid preparation step using a purifier. This provides the effect of removing impurities that may have been introduced into the mixed liquid.

Here, “purification” refers to a process that removes impurities and foreign substances derived from the styrene-based resin composition so that the concentration of the styrene-based polymer in the mixed liquid after the purification step is higher than the concentration of the styrene-based polymer in the mixed liquid before the purification step.

The purifier used in the present embodiment is not particularly limited as long as it is applicable to the mixed liquid. Pre-purification may also be performed before or during the devolatilization step described later, using a known purification mechanism.

The purification mechanism is not particularly limited, but it is preferable that it ensures uniform purification of each component in the mixed liquid. Specific examples of the purification mechanism include filtration, decantation, centrifugal separation, centrifugal sedimentation, screw decanter, strainer, screen mesh, or filter. The purification mechanism may be a single method or a combination of two or more methods. Among these, a purification mechanism capable of continuous processing (e.g., centrifugal separation, filtration, screw decanter, strainer, screen mesh, or filter) is preferable, and a purification mechanism combining centrifugal separation and filtration is more preferable. This enables the removal of impurities that may be mixed into the mixed liquid through centrifugal separation, thereby improving the time efficiency (e.g., processing speed) of filtration. The purification time, which refers to the time from introducing the mixed liquid into the purifier to obtain the purified mixed liquid through the purifier, is preferably about 1 second to 240 minutes.

The centrifugal separation used in the present embodiment is preferably performed using a centrifuge. The centrifuge includes a rotating body (basket) with holes or slits on the side thereof, which generates centrifugal force upon rotation to separate solids from liquids or separate substances with different specific gravities. The centrifuge preferably performs centrifugal separation within a range of 200 to 20,000 G of centripetal acceleration. The rotating body preferably has a vertical cross-sectional shape, including the rotation axis thereof, that has a substantially cylindrical or substantially truncated conical shape. Lightweight aggregates contained in the mixed liquid are supplied to the inside of the rotating body of the centrifuge, where the aggregates undergo centrifugal separation. As a result, they gather near the rotation axis of the rotating body as floating components and are separated from the rest of the mixed liquid.

As the centrifuge, for example, a disk-type centrifuge or a screw decanter is preferred.

In the present embodiment, to efficiently remove impurities that may be mixed into the mixed liquid, a filter (e.g., a bag, etc., made of nonwoven fabric or resin sheet) described later may be provided along the inner wall of the rotating body of the centrifuge. By providing a bag made of nonwoven fabric or resin sheet along the inner wall of the rotating body, the effects of both centrifugal sedimentation and centrifugal filtration can be achieved. As a result, impurities, etc., accumulated inside the rotating body can be removed together with the filter, nonwoven fabric, or resin sheet bag.

The filtration used in the present embodiment is preferably filtration using a filter. The filter may contain a filtration aid on the surface and/or inside of the filter. From the perspective of reducing scratches and particles, the pore size of the filter is preferably 1/10 or less, more preferably 1/20 or less, and even more preferably 1/30 or less of the average particle diameter of the filtration aid. From the perspective of preventing leakage of the filtration aid, the upper limit of the pore size of the filter is preferably 10 μm or less, more preferably 5 μm or less, and even more preferably 3 μm or less. Furthermore, from the perspective of improving the liquid permeation rate of the filter, the lower limit of the pore size of the filter is preferably 0.1 μm or more, more preferably 0.2 μm or more, and even more preferably 0.3 μm or more.

Examples of the filter materials include filter paper, polyethylene, polypropylene, polyethersulfone, polyphenylene sulfide, cellulose acetate, nylon, polycarbonate, Teflon® (Teflon is a registered trademark in Japan, other countries, or both), ceramics, and metal mesh.

The shape of the filter is not particularly limited, but from the perspective of ease of handling and reducing scratches and particles, a sheet-type, cylindrical-type, disk-type, or pleated-type filter is preferred.

The filtration conditions using the filter are not particularly limited. However, from the perspective of improving filtration accuracy and productivity, the differential pressure during filtration is preferably 0.008 to 10 MPa, more preferably 0.05 to 1 MPa. The number of filter stages is preferably 1 to 5, more preferably 1 to 3, from the perspective of improving filtration accuracy and productivity. The filtration rate is preferably 0.1 L/(min·m²) or more, more preferably 5 L/(min·m²) or more, from the perspective of improving filtration accuracy and productivity.

When a filter cloth is used as the filter, the air permeability of the filter can be 1 or more 3000 or less cm³/cm²·min or less, preferably more than 1 and less than 3000 cm³/cm²·min, and more preferably 20 or more and less than 2500 cm³/cm²·min.

The material of the filter cloth preferably has low solubility in solvents (for example, when 100 parts by mass of the solvent is added to 1 part by weight of the filter cloth, soaked at 40° C. for 1 hour, subjected to solid-liquid separation, and the filter cloth is recovered as a solid and vacuum dried at an ambient temperature of 100° C. for 1 hour, the weight change compared to the original weight if the filter cloth is less than 10%). Preferred materials include metals, nylon, fluororesins (such as polytetrafluoroethylene), PPS, PAEK (polyarylene ether ketone), polyester, polypropylene, and polyethylene. Stainless steel, nylon, fluororesins (such as polytetrafluoroethylene), PPS, and PAEK are more preferred.

The weaving method of the filter cloth may be plain weave, twill weave (also called diagonal weave), or satin weave, with twill weave being preferred.

In the purified mixed liquid obtained through the purifier, the content of the styrene-based polymer in the mixed liquid is preferably 5 mass % or more and 100 mass % or less, more preferably 5 mass % or more and 50 mass % or less relative to the total purified mixed liquid.

Additionally, in the purified mixed liquid obtained through the purifier, the content of impurities in the mixed liquid is preferably 30 mass % or less, more preferably 20 mass % or less relative to the total purified mixed liquid. The content of impurities in the purified mixed liquid is defined as the total content of the styrene-based polymer and the solvent subtracted from the total content of the mixed liquid. The purified mixed liquid may contain other resins that substantially do not contain styrene monomer units, such as olefinic resins, polyether-based resins, polyester-based resins, or polyamide-based resins, for example.

In the purification step or purifier in the present embodiment, if necessary, purification aids (commonly referred to as filtration aids) such as diatomaceous earth, perlite, powdered silica, powdered activated carbon, or aluminum silicate may be added to the mixed liquid prepared in the mixed liquid preparation step to improve purification efficiency. These aids not only assist in the removal efficiency of impurities (such as pigments, other resins, or other inorganic substances) or fine particles that are difficult to remove without aids in the purifier, but they also contribute to the removal and/or reduction of undesirable chemicals in the production and recovery of the styrene monomer from the mixed liquid through chemical and/or physical adsorption by the aids, as well as encapsulation and/or support by the aggregation of these aids. Here, encapsulation and/or support by the aggregation of aids refers to a mechanism in which specific components in the mixed liquid are surrounded by an aggregate of aid particles, creating a condition that inhibits diffusion into the mixed liquid.

The aid is preferably granular. The volume average particle size (D50) of the aid is preferably 5 μm to 100 μm, more preferably 10 μm to 50 μm, for example. The particle size distribution of the average particle diameter (D50) of the aid was measured using a laser diffraction particle size distribution analyzer (product name “SALD-2300” manufactured by Shimadzu Corporation), and the 50% value of the obtained particle size distribution was used to determine the volume average particle size (D50).

The amount of the aid added to the purifier is preferably 50 mass % or less, more preferably 0.001 mass % or more and 45 mass % or less, and even more preferably 0.1 mass % or more and 30 mass % or less, relative to the total weight of the mixed liquid. Furthermore, the amount of the aid is preferably 100 mass % or less, more preferably 0.01 mass % or more and 50 mass % or less, and even more preferably 0.1 mass % or more and 40 mass % or less, relative to the total weight of impurities in the mixed liquid.

As a method for adding the aid, the so-called body-feed method may be used, in which the aid described later is added during the mixing or kneading in the mixed liquid preparation step. Alternatively, the aid may be added to the mixed liquid after purification through the purifier. In this case, the method for mixing the aid into the mixed liquid and the method for mixing the mixed liquid containing the aid are not particularly limited, but a method that ensures uniform mixing of each component in the mixed liquid is preferred. If necessary, melt mixing or melt kneading may be used. Specific examples of the mixing or kneading mechanisms include a magnetic stirrer, Three-One Motor, single-screw extruder, twin-screw extruder, screw-type extruder, open roll, kneader, Banbury mixer, or internal mixer.

The time for mixing or kneading the purified mixed liquid with the aid is preferably about 1 to 240 minutes, more preferably 1 to 120 minutes, even more preferably 2 to 120 minutes, and still even more preferably 5 to 120 minutes.

The temperature for mixing or kneading the purified mixed liquid with the aid is preferably 0 to 260° C., more preferably 10 to 100° C., and even more preferably 20 to 80° C. Furthermore, when mixing, the pressure difference between atmospheric pressure and the pressure inside the mixing mechanism (or mixing apparatus) is preferably within ±500 hPa, more preferably within ±300 hPa. If necessary, the inside of the mixing mechanism (or mixing apparatus) may be replaced with an inert gas. Specific examples of the inert gas include nitrogen gas, argon gas, and carbon dioxide gas.

As another method for adding the aid, the purification may be performed by applying the aid or a dispersion of the aid in advance to the inside or outer surface of the purification mechanism and then bringing the mixed liquid into contact with it. As the dispersion, a substance that remains liquid at normal temperature and pressure and helps the aid to be uniformly dispersed is preferred. It is preferable that the solvent is selected from the solvents of the present embodiment. Examples of the solvent include one or more solvents selected from the group consisting of acetone, chloroform, methyl ethyl ketone, benzene, tetrahydrofuran, toluene, ethylbenzene, and styrene. The solvent may be used alone or as a mixed solvent containing two or more solvents.

In this step, specific examples of the purification mechanism include filtration, decantation, centrifugal separation, centrifugal sedimentation, screw decanter, and filter. The purification time, which refers to the time from introducing the mixed liquid into the purifier after the aid or the dispersion of the aid has been applied to the inside or outer surface of the purification mechanism, to obtain the purified mixed liquid through the purifier, is preferably about 1 second to 240 minutes, for example.

### (Devolatilization Step: S3)

The method for producing a styrene monomer of the present embodiment comprises a devolatilization step of devolatilizing the solvent components contained in the purified mixed liquid (including the solvent and any solvent contained in the raw material styrene-based resin composition) to obtain a fluid.

This step provides the effect of volatilizing low-molecular-weight components and removing the solvent.

In this specification, the product obtained by performing the devolatilization step on the purified mixed liquid is referred to as a fluid.

The devolatilization step in the present embodiment is not particularly limited as long as it is performed using an apparatus applicable to the mixed liquid. Preliminary devolatilization may also be performed before or during the thermal decomposition step described later, using a known devolatilization apparatus.

The devolatilization step is not particularly limited, but it is preferable that volatile components in the purified mixed liquid are uniformly removed. Specific examples of the devolatilization step include the use of conventional devolatilization apparatuses such as a flash drum, flash tank polymer heater, twin-screw devolatilizer, thin-film evaporator, or extruder, for example. Among them, a devolatilization apparatus with minimal retention zones is preferred.

The temperature of the devolatilization treatment in the devolatilization step (for example, the temperature inside the devolatilization apparatus) is typically about 100 to 280° C., and more preferably 190 to 260° C. The pressure for the devolatilization process (for example, the pressure inside the devolatilization apparatus) is typically about 0.13 to 5.0 kPa, preferably 0.13 to 4.5 kPa, and more preferably 0.13 to 4.0 kPa.

A preferred devolatilization step in the present embodiment includes, for example, a method in which the purified mixed liquid is heated to 100 to 280° C. inside the devolatilization apparatus while reducing the pressure inside the apparatus to remove volatile components, or a method in which volatile components are removed using an extruder or similar apparatus designed for volatilization removal.

For example, if the viscosity of the fluid needs to be adjusted, a residual solvent may remain as a result of the devolatilization step. Alternatively, a solvent may be added separately using the above-mentioned mixing or kneading mechanism for adjusting the viscosity. In this case, the solvent to be added separately may be preheated, and the temperature of the fluid to be adjusted is preferably about 50 to 280° C., more preferably 190 to 260° C.

The content of the solvent in the fluid is preferably 90 mass % or less, more preferably 50 mass % or less relative to the entire fluid.

Additionally, the content of the styrene-based polymer in the fluid is preferably 10 mass % or more and 100 mass % or less, more preferably 50 mass % or more and 100 mass % or less relative to the entire fluid.

The amount of impurities that may be contained in the fluid obtained through the devolatilization step is defined as the total amount of the styrene-based polymer and the solvent subtracted from the total amount of the fluid.

### <Solvent Recovery and Purification Step>

The method for producing a styrene monomer of the present embodiment may optionally include a step of recovering the solvent component that has been removed from the purified mixed liquid during the devolatilization step (S3) and purifying the solvent by distillation (solvent recovery and purification step). The solvent purified through this solvent recovery and purification step (i.e., so-called recycled solvent) may be reused in the mixed liquid preparation step (S1). Additionally, the residue other than the recycled solvent obtained from the solvent recovery and purification step may be blown down and used as fuel for the first thermal decomposition step (S4).

The distillation method can appropriately employ known distillation conditions according to the type of solvent used.

### (First Thermal Decomposition Step: S4)

The method for producing a styrene monomer of the present embodiment comprises a first thermal decomposition step of thermally decomposing the fluid prepared in the devolatilization step. Since the fluid with a high concentration of styrene-based polymers is used, it is decomposed into high-purity styrene monomer by heat.

The fluid only needs to contain a styrene-based resin composition containing a styrene-based polymer that includes styrene monomer units. Preliminary thermal decomposition may also be performed before or during the thermal decomposition step. Additionally, optional additive components described above may be added to the fluid if necessary, either before or during the thermal decomposition step.

The content of the styrene-based polymer in the fluid is preferably 10 mass % or more and 100 mass % or less, more preferably 50 mass % or more and 100 mass % or less relative to the entire fluid.

A method for thermally decomposing the fluid in the present embodiment may include, for example, filling the fluid into a thermal decomposition apparatus and performing the first thermal decomposition step by heating the fluid in an atmosphere with a temperature of 400 to 800° C., preferably 450 to 600° C., and more preferably 450 to 580° C. (e.g., the temperature inside the thermal decomposition apparatus). At this time, the temperature of the fluid before filling is preferably about 50 to 280° C., more preferably 190 to 260° C. Furthermore, the first thermal decomposition step may be performed by filling the fluid into the thermal decomposition apparatus that is preheated. When the thermal decomposition apparatus is preheated, the temperature inside the thermal decomposition apparatus is preferably 400 to 800° C., more preferably 450 to 600° C., while the temperature of the fluid before filling is preferably about 50 to 280° C., more preferably 190 to 260° C. Since the thermal decomposition temperature of styrene-based polymers (e.g., polystyrene, rubber-modified polystyrene-based resin, styrene-based copolymer resin) is about 330 to 380° C., setting the thermal decomposition apparatus within the above temperature range allows most of the resulting thermally decomposed vapor to become thermally decomposed products of styrene-based polymers. Additionally, performing the first thermal decomposition step on the fluid enables the removal of other resins, such as polyolefin-based resins, that may be contained in the raw material styrene-based resin composition containing a styrene-based polymer that includes styrene monomer units.

In the present embodiment, the first thermal decomposition step may be performed under reduced pressure or atmospheric pressure, but it is preferably performed under reduced pressure. Specifically, the reaction pressure in the first thermal decomposition step is preferably 1 to 100 kPa, more preferably 2 to 50 kPa, and even more preferably 3 to 30 kPa.

Since the first thermal decomposition step requires a temperature at which the material is heated and evaporated into gaseous raw material, performing it under reduced pressure has the effect of suppressing the formation of by-products during decomposition.

The thermal decomposition apparatus used in the present embodiment may be a known thermal decomposition apparatus. For example, the thermal decomposition apparatus in the present includes a thermal decomposition apparatus connected for the introduction of the fluid, a raw material supply pump that feeds the fluid, and a heating mechanism (such as a hot plate, electric heater, or hot air furnace) installed around the thermal decomposition apparatus. The fluid may also be stored in a storage tank provided before being connected to the thermal decomposition apparatus.

Additionally, the method of the thermal decomposition apparatus used in the first thermal decomposition step is not particularly limited, and known methods can be employed. For example, commonly used thermal decomposition methods such as external heating rotary kiln-type thermal decomposition apparatus and fluidized bed-type thermal decomposition apparatus are applicable.

Furthermore, if necessary, an apparatus for supplying a polymerization inhibitor may be used to intermittently or continuously supply the polymerization inhibitor, thereby suppressing the polymerization reaction of polymerizable components contained in the thermal decomposition liquid.

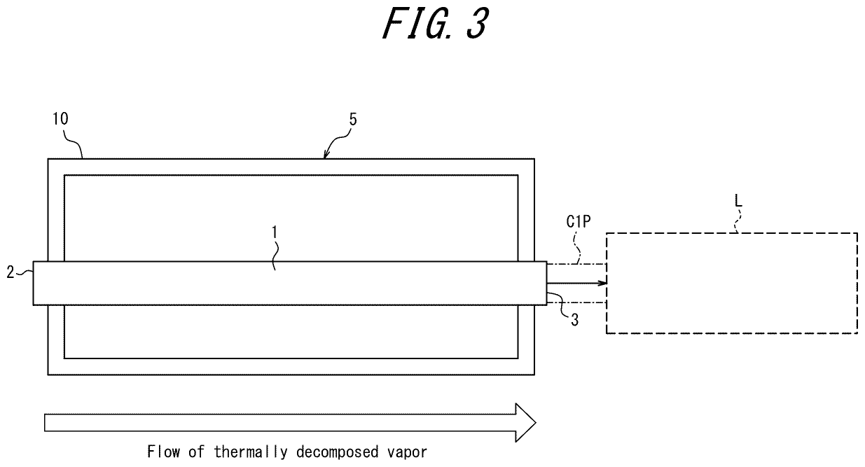

One example of the thermal decomposition apparatus in the present embodiment is the thermal decomposition apparatus illustrated in FIG. 3.

Specifically, the thermal decomposition apparatus 10 includes a cylindrical thermal decomposition section 1 having an opening 2 and an outlet 3 at the respective ends, and a thermal decomposition furnace 5 that surrounds the thermal decomposition section 1. Additionally, the outlet 3 is fluidly connected via a connection section C1P (e.g., a tube such as a glass tube) to a liquefaction apparatus L that cools the thermally decomposed vapor.

In one example illustrated in FIG. 3, the thermal decomposition furnace 5 (more specifically, the wall of the decomposition furnace 5) covers the cylindrical thermal decomposition section 1, forming a cavity between the decomposition furnace wall and the cylindrical thermal decomposition section 1, in which various heating mechanisms are installed. As an example of the heating mechanism, a heat transfer medium (heating gas) can be fed into the cavity of the thermal decomposition furnace 5, thereby heating the cylindrical thermal decomposition section 1 and controlling it to a certain temperature range. If the inlet for the heating medium (heating gas) fed into the cavity of the thermal decomposition furnace 5 is placed near the opening 2 where the fluid is introduced, the ambient temperature around the resin composition fed into the thermal decomposition section 1 increases rapidly. This rapid heating is preferable for suppressing the generation of thermal decomposition residue.

When the fluid is fed from the opening 2, the cylindrical thermal decomposition section 1 heated by the thermal decomposition furnace 5 allows the raw material composition to move along the process flow direction (indicated by the arrow) while being converted into thermally decomposed vapor. The raw material composition thermally decomposed into the thermally decomposed vapor is discharged from the outlet 3 in the state of thermally decomposed vapor, which is then transported through the connection section C1P (e.g., a tube) to the liquefaction apparatus L that cools the thermally decomposed vapor to prepare a (first) thermal decomposition liquid.

Additionally, in the thermal decomposition apparatus 10 in the present embodiment, vent holes (not illustrated) in the cylindrical thermal decomposition section 1 and a tube (not illustrated) that allows the vented thermally decomposed vapor to pass through may be provided to adjust the amount of the thermally decomposed vapor generated inside the cylindrical thermal decomposition section 1 and discharge the vapor to the outside. The tube may be extended near the outlet 3 of the thermal decomposition section 1, for example. This configuration allows the vented thermally decomposed vapor flowing out from the vent holes (not illustrated) to be recovered through the tube, to which a valve or similar control mechanism may be provided, if necessary.

The term “thermal decomposition” in this specification refers to chemical decomposition performed by heating organic materials in the absence of oxygen or other substances.

In the first thermal decomposition step, the fluid is heated to obtain the first thermal decomposition liquid. More specifically, in the first thermal decomposition step, it is preferable to cool the thermally decomposed vapor containing the styrene monomer to obtain the first thermal decomposition liquid. Specifically, the thermally decomposed vapor generated by heating the fluid in the thermal decomposition apparatus is cooled to obtain the first thermal decomposition liquid containing the styrene monomer. The cooling temperature for the thermally decomposed vapor is preferably the temperature at least 30° C. lower than the boiling point T_{sb} (° C.) of the styrene monomer, and more preferably −20° C. to 80° C.

Since olefinic hydrocarbon compounds with boiling points of −30° C. or lower (such as ethylene and propylene) do not liquefy or hardly liquefy, setting the cooling temperature within this range allows effective removal of low-boiling-point monomers, which are decomposition products of polyolefin-based resins that may be contained in the styrene-based resin composition. Additionally, in the cooling process, the fraction with a boiling point below T_{sb} (° C.) of the styrene monomer may be separately stored and used as a combustion raw material for the thermal decomposition apparatus.

Furthermore, the thermally decomposed vapor generated by heating the fluid in the first thermal decomposition step may be liquefied in the liquefaction apparatus into the first thermal decomposition liquid, which contains high-boiling-point components cooled to a temperature not lower than the boiling point T_{sb} (° C.) of the styrene monomer and not higher than 450° C., and this liquid may be dripped back into the thermal decomposition apparatus to undergo thermal decomposition again. Additionally, the generated thermally decomposed vapor may be cooled using the liquefaction apparatus to a temperature not lower than −30° C. and not higher than the boiling point T_{sb} (° C.) of the styrene monomer, thereby liquefying the vapor and obtaining it as a part of a second thermal decomposition liquid, which will be described later.

For cooling the thermally decomposed vapor, it is preferable to use a liquefaction apparatus. A known liquefaction apparatus can be used. For example, various heat-dissipating solvents such as water and a cooling tube utilizing heat dissipation effects of a heat dissipation element made of a metal and other inorganic materials, which can cool to the cooling temperature, can be employed.

Furthermore, if necessary, a reforming apparatus for modifying the components of the thermally decomposed vapor (including, for example, dechlorination, adsorption of odorous or coloring components, removal of acidic or basic components, and heat treatment) may be fluidly connected between the thermal decomposition apparatus and the liquefaction apparatus.

Additionally, an analysis step may be provided as needed to analyze the first thermal decomposition liquid. This analysis step can evaluate characteristics, such as the styrene monomer concentration in the first thermal decomposition liquid, the temperature of the first thermal decomposition liquid, and the viscosity of the first thermal decomposition liquid.

### <Deposits in Cooling Tube>

In our efforts to develop the method for producing a styrene monomer of the present embodiment, it was demonstrated that deposits may be formed inside the cooling tube during the cooling in the first thermal decomposition step. When such deposits accumulate in the cooling tube, the thermal efficiency of the thermal decomposition apparatus decreases, which in turn lowers the thermal decomposition efficiency, leading to potential blockages of piping or reduced processing capacity. Additionally, these deposits may decrease the cooling efficiency when the thermally decomposed vapor generated by the thermal decomposition of the fluid is cooled to obtain the first thermal decomposition liquid.

Since the impurities contained in the fluid leading up to the first thermal decomposition step are substantially reduced in the method for producing a styrene monomer of the present embodiment, it is expected that the formation of deposits in the cooling tube will also be reduced.

In the present embodiment, it is preferable that the first thermal decomposition step includes heating the fluid to obtain thermally decomposed vapor and then cooling the thermally decomposed vapor to produce the first thermal decomposition liquid. More specifically, the fluid is heated at a certain temperature and at a certain pressure using a thermal decomposition apparatus to generate thermally decomposed vapor containing the styrene monomer, which is then cooled to obtain the first thermal decomposition liquid containing the styrene monomer. The cooling temperature of the thermally decomposed vapor is preferably at least −30° C. lower than the boiling point T_{sb} (° C.) of the styrene monomer, and more preferably −20° C. to 80° C.

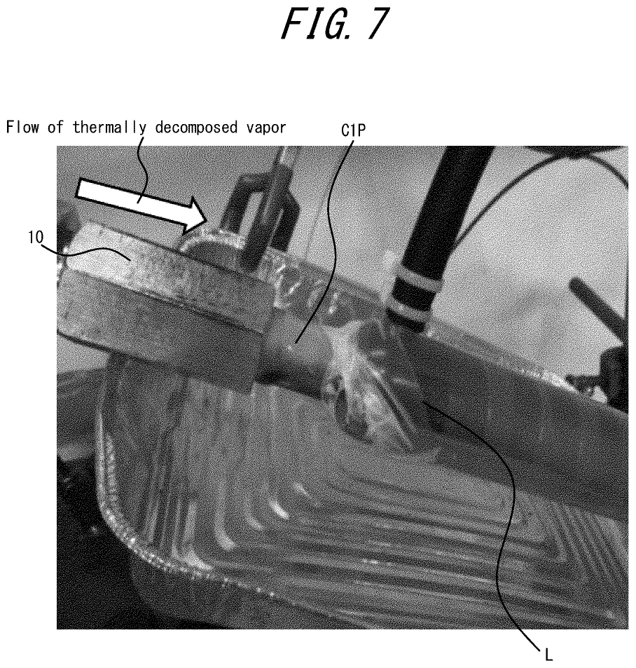

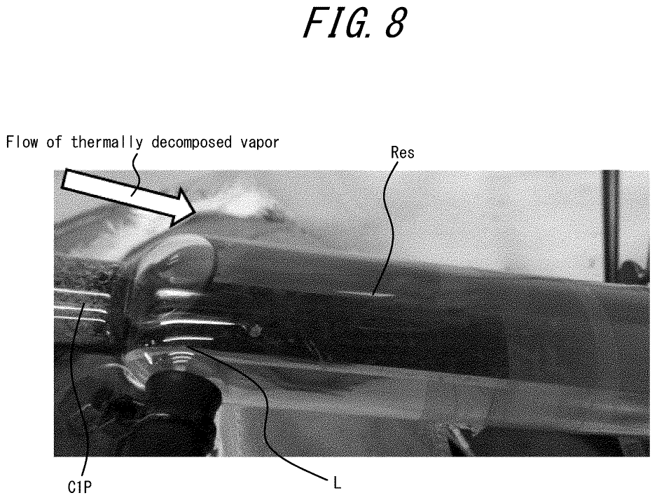

For example, when the thermal decomposition apparatus illustrated in FIG. 3 in the first thermal decomposition step is used in the first thermal decomposition step, the outlet 3 is fluidly connected to the liquefaction apparatus. Therefore, it has been demonstrated that when the resulting thermally decomposed vapor containing the styrene monomer is injected into the liquefaction apparatus, deposits may be formed inside the pipe (also referred to as the inner wall of the cooling pipe or heat exchange) in the liquefaction apparatus from the connecting section (commonly referred to as connection pipe) that fluidly connects the thermal decomposition apparatus and the liquefaction apparatus (see FIG. 7 or FIG. 8). Through analyses of these deposits, it was found that when certain components contained in the obtained first thermal decomposition liquid (hereinafter referred to as “deposit-inducing substances”) is present in a large amount, the quantity of deposits on the inner wall of the cooling pipe or heat exchanger (including the connecting section that fluidly connects the thermal decomposition apparatus and the liquefaction apparatus to the piping inside the liquefaction apparatus) increases. Furthermore, it was discovered that by setting the type of solvent used and the conditions of the purification step to certain conditions, the amount of deposit-inducing substances and deposits on the inner wall of the cooling pipe or heat exchanger was reduced.

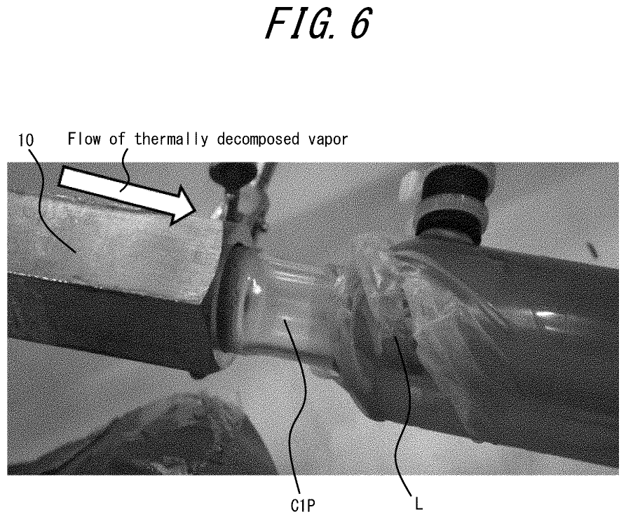

More specifically, when the styrene monomer in the present is produced using the liquefaction apparatus L connected via the connecting section C1P (glass tube) that connects the thermal decomposition section 1 of the thermal decomposition apparatus 10 to the thermal decomposition apparatus 10 illustrated in FIG. 3, it was demonstrated from the image in FIG. 6 that the connecting section C1P (glass tube) was transparent just before the start of the first thermal decomposition step. However, during thermal decomposition operation, the connecting section C1P (glass tube) gradually became clouded, and a milky-white film-like deposit was observed on the inner wall of the connecting section C1P (see FIG. 7). Furthermore, as the thermal decomposition operation continued, a black substance Res was found to have adhered over the entire inner walls of the connecting section C1P and liquefaction apparatus L, causing almost complete blockage (see FIG. 8).

In the present embodiment, the first thermal decomposition step is a step comprising heating the fluid to generate thermally decomposed vapor containing the styrene monomer, followed by cooling the thermally decomposed vapor to produce the first thermal decomposition liquid, wherein the concentration of deposit-inducing substances in the first thermal decomposition liquid is preferably less than 0.2 mass %.

As described above, the analyses of the deposits on the inner wall surface of the cooling tube or heat exchanger revealed a correlation between the amount of deposit-inducing substances in the first thermal decomposition liquid and the amount of deposits on the inner wall surface of the cooling tube or heat exchanger. However, by reducing the concentration of deposit-inducing substances in the first thermal decomposition liquid to less than 0.2 mass %, the amount of deposits on the inner wall surface of the cooling tube or heat exchanger can be reduced. Moreover, as discussed in the examples and comparative examples described later, when the concentration of deposit-inducing substances in the first thermal decomposition liquid was less than 0.2 mass %, the glass tube of the connection section C1P remained transparent, as illustrated in FIG. 6, and no thin milky-white deposits were observed on the inner wall of the cooling tube or heat exchanger. Conversely, when the concentration of deposit-inducing substances in the first thermal decomposition liquid was 0.2 mass % or higher, the glass tube of the connection section C1P became clouded or thin milky-white deposits were observed on the inner wall of the cooling tube or heat exchanger, as illustrated in FIG. 7. Furthermore, when the operation was continued with these deposits left on the inner walls, black deposits (Res) adhered to the entire inner walls of the connection section C1P and liquefaction apparatus L, causing almost complete blockage.

The concentration of deposit-inducing substances in the first thermal decomposition liquid may be 0 to less than 0.2 mass %, preferably more than 0 mass % and less than 0.19 mass %, and more preferably more than 0 mass % and less than 0.18 mass %. Among these, the amount of deposit-inducing substances in the first thermal decomposition liquid below 0.2 mass % is preferred in view of long-term continuous operation.

In this specification, the term “deposit-inducing substances” refers to substances predominantly composed of the thermal decomposition components of impurities and include inorganic salts (particularly those containing elements from periods 2 to 5 of groups 1 to 17 in the periodic table) or low-molecular-weight organic compounds with a melting point of 100° C. or lower and a molecular weight (or number average molecular weight) of 10 to 1000 (particularly those with a melting point of 40° C. or lower as a single compound), as well as resins such as polyethylene, polypropylene, polyacetal (particularly polyoxymethylene), polyamide (particularly polyamide 6 and polyamide 66), polyvinyl chloride, polyester (particularly polyethylene terephthalate and polybutylene terephthalate), polyether, and polyphenylene ether (particularly modified polyphenylene ether), and particularly resins with a weight average molecular weight (Mw) of less than 100,000.

### (Recovery Step: S5)

The method for producing a styrene monomer of the present preferably optionally includes a recovery step of recovering the styrene monomer from the first thermal decomposition liquid prepared in the first thermal decomposition step.

This step allows impurities and low-volatility substances (such as solvents and olefin-based hydrocarbon compounds) to be removed via the devolatilization step, thereby enabling the recovery of the styrene monomer.

Furthermore, it is preferable that this recovery step employs distillation to recover the styrene monomer from the first thermal decomposition liquid. In other words, for example, the recovery step is preferably a distillation recovery step of recovering the styrene monomer from the first thermal decomposition liquid containing the styrene monomer through distillation. It is preferable that the distillation recovery step is conducted in two or more stages, using two or more distillation columns. Additionally, it is preferred that these two or more distillation columns are filled with an inert gas (such as nitrogen or noble gases) supplied from an inert gas source, replacing the air inside the distillation columns with the inert gas before distillation.

In the distillation recovery step, the distillation temperature is preferably within a range of 50 to 200° C., more preferably 50 to 150° C., for example. For example, when the first thermal decomposition liquid is distilled in two stages using two distillation columns, the distillation temperature for the first stage is in the range of preferably 50 to 200° C., more preferably 50 to 150° C., for example. The distillation temperature for the second stage is in the range of preferably 50 to 200° C., more preferably 50 to 150° C., for example.

The distillation pressure (e.g., the pressure inside the distillation column) in the distillation recovery step is preferably 10 to 70 Torr, more preferably 20 to 60 Torr.

When the first thermal decomposition liquid is distilled under these conditions, it is separated into a fraction with a high concentration of the styrene monomer (commonly referred to as “light components”) and a fraction with a lower concentration of the styrene monomer than the aforementioned fraction (commonly referred to as “heavy components”)

For example, in the two-stage distillation process using two distillation columns, the light components are composed of low-boiling-point components (including toluene, for example) while the heavy components are composed of high-boiling-point components (including the styrene monomer, for example), in the first distillation column. After the light components are exhausted, the heavy components are preferably distilled again in the second-stage distillation column, thereby separating the fraction containing the styrene monomer with a high concentration from the fraction with a lower concentration of the styrene monomer than the aforementioned fraction, thus recovering the styrene monomer. In the distillation recovery step, a polymerization inhibitor may be used to prevent the polymerization of the styrene monomer.

The first thermal decomposition liquid only needs to contain the styrene monomer, and a preliminary thermal decomposition may be performed before or during the recovery step. Furthermore, in the recovery step during and/or after the first thermal decomposition step, optional additive components may be added to the fluid as needed to suppress the polymerization reaction of the produced styrene monomer.

In the present embodiment, a known recovery method can be used for recovering the styrene monomer. For example, a rectification column capable of rectifying the first thermal decomposition liquid to separate low-boiling-point components such as benzene or toluene from crude styrene monomer (the styrene monomer with a purity of 90% or less) may be fluidly connected. Additionally, the rectification column may be fluidly connected to a distillation column for further rectification of the crude styrene monomer to increase its purity. If necessary, a dechlorination apparatus for dechlorinating the components in the thermal decomposition liquid may be fluidly connected between the thermal decomposition apparatus and the rectification column.

### (First Distillation Step: S6)

The method for producing the styrene monomer of the present preferably includes, as needed, distilling the first thermal decomposition liquid to separate it into a first fraction containing the styrene monomer and a second fraction having a lower styrene monomer concentration than that in the first fraction. As a result, the second fraction containing the styrene monomer with a low concentration can be recovered, or the first fraction, which contains the styrene monomer, styrene dimer, and styrene trimer with higher concentrations than the second fraction, can be recovered. Since the fraction containing the styrene monomer with a high concentration can be recovered, the yield of the styrene monomer can be increased.

In other words, when the styrene-based resin composition is thermally decomposed during the first thermal decomposition step, by-products such as styrene dimer and styrene trimer may also be included in the first thermal decomposition liquid. Therefore, if these styrene dimer and styrene trimer can be effectively utilized, the yield of the styrene monomer can be further improved.

As a method for distilling the first thermal decomposition liquid to separate it into a first fraction containing the styrene monomer, styrene dimer, and styrene trimer, and a second fraction having a lower styrene monomer concentration than that in the first fraction, distillation using a distillation column is preferable.

Therefore, the method for producing the styrene monomer of the present preferably comprises an optional first distillation step of distilling the first thermal decomposition liquid to separate it into a first fraction containing the styrene monomer, styrene dimer, and styrene trimer, and a second fraction having a lower styrene monomer concentration than that in the first fraction.

The first distillation step is preferably performed in one or more stages using one or more distillation columns. Moreover, the one or more distillation columns are preferably filled with an inert gas (e.g., nitrogen and noble gases) supplied from an inert gas supply source, thereby replacing the air inside the distillation column for distillation.

The distillation temperature in the first distillation step is in the range of preferably 50 to 200° C., more preferably 50 to 150° C., for example. For example, when the thermal decomposition liquid is distilled in a single stage using a single distillation column, the distillation temperature in this stage is in the range of preferably 50 to 200° C., more preferably 50 to 150° C., for example.

The pressure of the distillation atmosphere (e.g., the pressure inside the distillation column) in the first distillation step is preferably 10 to 70 Torr, more preferably 20 to 60 Torr.

When the first thermal decomposition liquid is distilled under these distillation conditions, it is separated into a first fraction (commonly referred to as high-boiling-point component) containing the styrene monomer, styrene dimer, and styrene trimer, and a second fraction (commonly referred to as low-boiling-point component) having a lower styrene monomer concentration than that in the first fraction.

The difference in the styrene monomer concentration between the first fraction and the second fraction is preferably such that the styrene monomer concentration in the first fraction is at least about twice that in the second fraction.

For example, when the first thermal decomposition liquid is distilled in a single stage using a single distillation column in the first distillation step, the low-boiling components are composed of low-boiling-point components (e.g., toluene) while the high-boiling components are composed of the styrene monomer, styrene dimer, styrene trimer, and the like, in the distillation column. In the first distillation step, a polymerization inhibitor may be used to prevent the polymerization of the styrene monomer.

### (Second Distillation Step (S7))

The method for producing the styrene monomer of the present preferably includes, as necessary, distilling the first fraction to separate it into a third fraction containing the styrene monomer and a fourth fraction having a lower styrene monomer concentration than that in the third fraction. As a result, the styrene monomer can be recovered from the third fraction, which contains the styrene monomer at a high concentration. Alternatively, the fourth fraction, which contains styrene dimer and styrene trimer at high concentrations than the third fraction, can be recovered. This allows for the recovery of a fraction containing the styrene monomer with a high concentration, thereby improving the yield of the styrene monomer. When the styrene-based resin composition is thermally decomposed in the first thermal decomposition step, by-products such as styrene dimer and styrene trimer may also be contained in the first thermal decomposition liquid. Therefore, if these styrene dimer and styrene trimer can be effectively utilized, the yield of the styrene monomer can be further improved.

As a method for distilling the first fraction to separate the first fraction into a fourth fraction containing styrene dimer and styrene trimer and a third fraction having a higher styrene monomer concentration than that in the fourth fraction, distillation using a distillation column is preferable.

Accordingly, the method for producing the styrene monomer of the present embodiment preferably comprises an optional second distillation step of distilling the first fraction to separate it into a fourth fraction containing styrene dimer and styrene trimer and a third fraction having a higher styrene monomer concentration than that in the fourth fraction.

In this second distillation step, it is preferable to perform the distillation in one or more stages using one or more distillation columns. Moreover, these one or more distillation columns are preferably filled with an inert gas (e.g., nitrogen and noble gases) supplied from an inert gas supply source, thereby replacing the air inside the distillation column during distillation.

Additionally, the distillation temperature in the second distillation step is in the range of preferably 50 to 200° C., more preferably 50 to 150° C., for example. For example, when distilling the fourth fraction in a single stage using a single distillation column in the second distillation step, the distillation temperature in this stage is in the range of preferably 50 to 200° C., more preferably 50 to 150° C., for example. The pressure of the distillation atmosphere (e.g., the pressure inside the distillation column) in the second distillation step is preferably 10 to 70 Torr, more preferably 20 to 60 Torr.

When the first fraction is distilled under these distillation conditions, it is separated into a fourth fraction (commonly referred to as high-boiling-point components) containing styrene dimer and styrene trimer and a third fraction (commonly referred to as low-boiling-point components) having a higher styrene monomer concentration than that in the first fraction.

The difference in the styrene monomer concentration between the third fraction and the fourth fraction is preferably such that the styrene monomer concentration in the third fraction is at least about twice that in the fourth fraction.

For example, in the second distillation step, when distilling the fourth fraction in a single stage using a single distillation column, the low-boiling fractions are composed of low-boiling-point components (e.g., the styrene monomer) while the high-boiling components are composed of styrene dimer, styrene trimer, and the like.

In the second distillation step, a polymerization inhibitor may be used to prevent the polymerization of the styrene monomer.

Since the fraction with the highest styrene monomer purity is the third fraction, it is preferable to recover this third fraction as the styrene monomer.

### (Second Thermal Decomposition Step (S8))

The method for producing the styrene monomer of the present embodiment preferably further comprises an optional second thermal decomposition step of thermally decomposing the fourth fraction again to produce a second thermal decomposition liquid. This allows for the thermal decomposition of the fourth fraction containing styrene dimer and styrene trimer, leading to further production of the styrene monomer and thereby improving the yield of the styrene monomer.

As a method for thermally decomposing the fourth fraction in the present embodiment, for example, after the fourth fraction is filled into a thermal decomposition apparatus, a second thermal decomposition step of heating the fourth fraction in an atmosphere at a temperature (e.g., the temperature inside the thermal decomposition apparatus) of 300 to 700° C., preferably 320 to 600° C., more preferably 350 to 500° C. may be performed. At this time, the temperature of the fourth fraction to be filled is preferably about 0 to 300° C., more preferably 25 to 280° C., even more preferably 25 to 250° C., and still even more preferably 25 to 150° C. Furthermore, a second thermal decomposition step of filling the fourth fraction into a thermal decomposition apparatus that is preheated may be performed.

In this case, when the thermal decomposition apparatus is preheated, the temperature inside the thermal decomposition apparatus is preferably 300 to 700° C., more preferably 320 to 600° C., and even more preferably 350 to 500° C., and the temperature of the fourth fraction to be filled is preferably about 0 to 300° C., more preferably 25 to 280° C., even more preferably 25 to 250° C., and still even more preferably 25 to 150° C. Since the thermal decomposition temperature of styrene dimer and styrene trimer contained in the fourth fraction in the atmospheric pressure is about 200 to 700° C., setting the temperature of the thermal decomposition apparatus within this range allows for the majority of the resulting thermally decomposed vapor to be the thermally decomposed products of styrene dimer and styrene trimer. This improves the yield of the styrene monomer.

In the present embodiment, the second thermal decomposition step may be performed under reduced pressure or atmospheric pressure, but it is preferably performed under reduced pressure. Specifically, the reaction pressure in the second thermal decomposition step is preferably 1 to 202 kPa, more preferably 7 to 101 kPa, even more preferably 20 to 101 kPa, and still even more preferably 20 to 80 kPa.

Since the second thermal decomposition step requires a temperature at which the material is heated and evaporated into gaseous raw material, carrying out the step under reduced pressure provides the effect of reducing by-products from decomposition.

Additionally, in the second thermal decomposition step, when the fourth fraction is heated to produce the second thermal decomposition liquid, it is preferable to cool the resulting thermally decomposed vapor to obtain the second thermal decomposition liquid. Specifically, the thermally decomposed vapor generated by heating the fourth fraction in the thermal decomposition apparatus is cooled to produce the second thermal decomposition liquid containing the styrene monomer. The cooling temperature for the thermally decomposed vapor is preferably the temperature at least 30° C. lower than the boiling point T_{sb} (° C.) of the styrene monomer, and more preferably −20° C. to 80° C.

The cooling may employ a known method similar to that used in the first thermal decomposition step.

### (Third Distillation Step (S9))

The method for producing the styrene monomer of the present embodiment preferably includes, as necessary, distilling the second thermal decomposition liquid to separate it into a fifth fraction containing the styrene monomer and a sixth fraction having a lower styrene monomer concentration than that in the fifth fraction. This improves the yield of the styrene monomer.

As a method for distilling the second thermal decomposition liquid to separate it into the fifth fraction containing the styrene monomer and the sixth fraction having a lower styrene monomer concentration than that in the fifth fraction, distillation using a distillation column is preferable.

Accordingly, the method for producing the styrene monomer of the present embodiment preferably comprises an optional third distillation step of distilling the second thermal decomposition liquid to separate it into a fifth fraction containing the styrene monomer and a sixth fraction having a lower styrene monomer concentration than that in the fifth fraction.

The third distillation step is preferably performed at least once using one or more distillation columns (commonly referred to as one-stage or more distillation). Additionally, the distillation column is preferably filled with an inert gas (e.g., nitrogen and noble gases) supplied from an inert gas supply source, thereby replacing the air inside the distillation column with the inert gas for distillation.

The distillation temperature in the third distillation step is in the range of preferably 50 to 200° C., more preferably 50 to 150° C., for example.

The distillation atmosphere pressure (e.g., the pressure inside the distillation column) in the third distillation step is preferably 8 to 70 Torr, more preferably 9 to 60 Torr.

By distilling the second thermal decomposition liquid under the above distillation conditions, the liquid is separated into a fifth fraction containing the styrene monomer (commonly referred to as low-boiling-point components) and a sixth fraction having a lower styrene monomer concentration than that in the fifth fraction (commonly referred to as high-boiling-point components).

The difference between the styrene monomer concentration in the fifth fraction and the styrene monomer concentration in the sixth fraction is preferably such that the styrene monomer concentration in the fifth fraction is about at least twice that in the sixth fraction.

Furthermore, in the third distillation step, a polymerization inhibitor may be used to prevent the polymerization of the styrene monomer.

### (Recycling Step (S10))

The method for producing the styrene monomer of the present embodiment preferably includes an optional recycling step of recovering the styrene monomer from the fifth fraction. This improves the yield of the styrene monomer.

The recycling step preferably comprises, for example, a step (I) of recovering the styrene monomer using the fifth fraction as a part of the first thermal decomposition liquid, and a step (II) of recovering the styrene monomer by distilling the fifth fraction separately from the first thermal decomposition liquid.

The details of the above-described section on (Recovery Step: S5) are applied to the step (I) of recovering the styrene monomer using the fifth fraction as a part of the first thermal decomposition liquid.

Additionally, the details of the above-described sections on (First Distillation Step: S6) and/or (Second Distillation Step: S7) and/or (Third Distillation Step: S9) are applied to the step (II) of recovering the styrene monomer by distilling the fifth fraction separately from the first thermal decomposition liquid.

### (Production Apparatus for Styrene Monomer)

The present disclosure relates to a production apparatus for styrene monomer comprising a dissolver for preparing a mixed liquid where a styrene-based resin composition containing a styrene-based polymer that includes styrene monomer units and a solvent are mixed, a purifier for purifying the mixed liquid, a devolatilization apparatus for devolatilizing the purified mixed liquid to obtain a fluid, a thermal decomposition apparatus for thermally decomposing the fluid, and a liquefaction apparatus for cooling thermally decomposed vapor obtained by the thermal decomposition apparatus to produce a first thermal decomposition liquid.

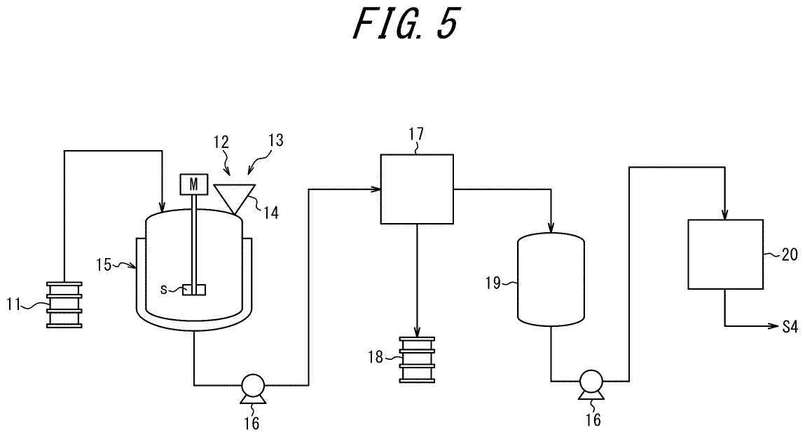

Hereinafter, an example of the production apparatus for styrene monomer will be described with reference to FIG. 5. FIG. 5 illustrates an apparatus incorporating the method for producing the styrene monomer of the present embodiment.

Specifically, the production apparatus for styrene monomer of the present includes a dissolver 15, which is a recessed container in which a raw material styrene-based resin composition 12, a filtration aid 13 optionally blended as needed, and a solvent 11 are charged and mixed, a purifier 17 for purifying the mixed liquid prepared by the dissolver 15, a devolatilization apparatus 20 for devolatilizing the purified mixed liquid prepared by the purifier 17 to prepare a fluid, a thermal decomposition apparatus 10 (FIG. 3) for thermally decomposing the fluid, and a liquefaction apparatus L (FIG. 3) for cooling thermally decomposed vapor obtained by the thermal decomposition apparatus 10 (FIG. 3) to produce a first thermal decomposition liquid, wherein these components are fluidly connected.

An example of the production of styrene monomer using this production apparatus for styrene monomer is as follows.

The raw material styrene-based resin composition 12 and the optionally added filtration aid 13 are charged into the dissolver 15, which is a recessed container, via a hopper 14. The solvent 11 is also charged into the dissolver 15, which is a recessed container. A rotating shaft S with stirring blades attached to one end is provided in the dissolver 15, and a motor (M), serving as a power source, is attached to the other end of the rotating shaft. Hence, when the rotating shaft S is rotated, the raw material styrene-based resin composition 12, the optionally added filtration aid 13, and the solvent 11 are mixed to prepare a mixed liquid inside the dissolver 15. Furthermore, since the dissolver 15 is fluidly connected to the purifier 17 via a pipe attached to the bottom or side of the dissolver 15, the mixed liquid prepared inside the dissolver 15 is pumped to the purifier 17 by a pump 16. As the purifier 17, a centrifugal separator with a filter provided inside a rotating body, as described above, can be used. This allows impurities in the mixed liquid to be removed and discharged to the outside as impurity cake 18. On the other hand, the purified mixed liquid, from which the impurities have been removed, is charged into a tank 19. Since the tank 19 is fluidly connected to the devolatilization apparatus 20 via a pipe, the purified mixed liquid charged into the tank 19 is pumped to the devolatilization apparatus 20 by a pump 16. Thereafter, the solvent and other components are removed by the devolatilization apparatus 20 to produce a fluid. The fluid is then pumped, for example, to the thermal decomposition apparatus illustrated in FIG. 3, where the first thermal decomposition Step (S4) and the recovery Step (S5) for recovering the styrene monomer from the first thermal decomposition liquid are performed.

### EXAMPLES

### [Measurement and Evaluation Method]

Measurements and evaluations of the physical properties of the resin composition obtained in each of the examples and comparative examples were performed based on the following methods.

### <Evaluation Method of Solvent in Mixed Solution Preparation Step>

In the mixed liquid preparation step of the present embodiment, the solubility of solvents and the styrene-based resin composition can be evaluated using GPPS pellets. Various solvents were evaluated as follows: 1.0 g of GPPS pellets and each solvent were added to a 100 mL screw vial to give a given mass percentage concentration, and they were mixed to prepare a mixed liquid using a shaker at 25° C. for 1 hour. If solid residues with a long side of 5 mm or more remained, the mixture was deemed unsuitable for preparing a mixed liquid (“Bad”), whereas if no solid residues with a long side of 5 mm or more remained, the solvent was deemed suitable for preparing a mixed liquid (“Good”). Table 1-1 lists the solvents used, the mass percentages thereof, and the evaluation results of whether the solvents were suitable for preparing a mixed liquid or not.

### <Thermal Decomposition Apparatus and Procedure Used in Examples and Comparative Examples>

### Examples 1 to 21

In Examples 1 to 21 and Comparative Examples 1 to 5, thermal decomposition experiments were conducted using a small reactor for experimental purposes (small laboratory-scale thermal decomposition apparatus). However, the size of the apparatus is not particularly limited as long as it does not deviate from the intended purpose of the experiment. The specific procedure is as follows.

A styrene-based resin composition containing a styrene-based polymer including styrene monomer units was charged into a SUS-made reaction vessel, placed inside a cast-in heater, and fitted with a SUS-made cover with a branch pipe. After bolts were tightened with a wrench, a dedicated adapter with an O-ring was attached to the branch pipe, and a glass-made Liebig condenser, a distillation adapter with a branch for vacuum hose attachment, and a round-bottom flask for collecting a thermal decomposition liquid were each attached with silicone grease applied. Then, a three-way cock was attached on the branch for the vacuum hose connection, and a nitrogen balloon and a vacuum pump were connected. The temperature was monitored using thermocouples installed inside the cast-in heater and the reaction vessel. Thermal decomposition was performed at 450° C. under decompressed pressure by setting the vacuum pump to 34 hPa while feeding the coolant at −10° C. and adjusting the output to the cast-in heater. The resulting thermally decomposed vapor was liquefied in the Liebig condenser through which the coolant flowed, and the thermal decomposition liquid was collected in the round-bottom flask.

### <Residue Rate>

The residue rate (A) in the method for producing a styrene monomer of the present embodiment can be calculated based on the crude residue rate (B), which was determined from the weight ratio of the styrene-based resin composition charged into the thermal decomposition apparatus to the solid residue remaining after thermal decomposition, as well as the thermogravimetric weight loss rate (C) measured under a nitrogen atmosphere and the thermogravimetric weight loss rate (D) measured under an air atmosphere. The residue rate (A), crude residue rate (B), thermogravimetric weight loss rate (C), and thermogravimetric weight loss rate (D) were calculated according to the following formula.

In this evaluation, a residue rate (A) of 0.4% or less is rated as “Excellent,” while 0.35% or less is rated as “Good.” If the residue rate (A) exceeds 0.5%, it may lead to a decrease in the thermal efficiency of the thermal decomposition apparatus, resulting in a reduction in thermal decomposition efficiency, pipe clogging, and decreased processing capacity. If the weight of the solid residue remaining after the thermal decomposition of the charged styrene-based resin composition is zero, the crude residue rate (B) becomes 0, and both the thermogravimetric weight loss rate (C) and thermogravimetric weight loss rate (D) are considered to be 0.

$$Residue rate ( A ) = ( crude residue rate ( B ) \times ( thermogravimetric weight loss rate ( C ) - thermogravimetric weight loss rate ( D ) ) + crude residue rate ( B ) \times thermogravimetric weight loss rate ( D ) ) / 100$$

### <Thermogravimetric Weight Loss Rate (%)>

The thermogravimetric weight loss rate (%) was measured using a TGA apparatus (TGA-50) manufactured by Shimadzu Corporation, with TA60-WS as the measurement software. A 10 mg sample of the crude residue obtained from the thermal decomposition experiment was placed in a deep-bottom aluminum pan. The measurement was conducted under a nitrogen or dry air flow of 20 mL/min using a condition program with a heating rate of 20° C./min, from 25° C. to 550° C., followed by holding at 550° C. for 60 minutes. The thermogravimetric weight loss rates (C) and (D) were calculated from the weight difference between the initial measurement point at 25° C. and the final measurement point after the heating program at 550° C. for 60 minutes.

$$Thermogravimetric weight loss rate ( C ) \% = ( remaining weight in the deep - bottom aluminum pan after measurement under nitrogen condition / sample weight measured in the deep - bottom aluminum pan before the start of the measurement ) \times 100$$

$$Thermogravimetric weight loss rate ( D ) \% = ( remaining weight in the deep - bottom aluminum pan after measurement under air condition / sample weight measured in the deep - bottom aluminum pan before the start of the measurement ) \times 100$$

### <Evaluation Method of Deposits>

The first thermal decomposition step in the present is a step of refining a mixed liquid in which a styrene-based resin composition containing a styrene-based polymer that includes styrene monomer units and a solvent are mixed, devolatilizing it to obtain a fluid, heating the fluid to generate thermally decomposed vapor containing the styrene monomer, and then cooling the thermally decomposed vapor to produce a first thermal decomposition liquid. During this process, deposits were observed on the inner wall surface of the heat exchange of the liquefaction apparatus used for cooling the thermally decomposed vapor (commonly referred to as the surface of the cooling tube). Samples with thick deposits confirmed on the surface were rated as “Poor” (e.g., the state in FIG. 8), samples with thin film-like deposits in a small amount were rated as “Fair” (e.g., the state in FIG. 7), and samples with no observable deposits were rated as “Excellent” (e.g., the state in FIG. 6). These results were recorded under the category of the presence of deposits in Tables 1-2 to 1-5.

The presence of deposits in the cooling tubes leads to a decrease in the thermal efficiency of the thermal decomposition apparatus, which in turn lowers the overall thermal decomposition efficiency, increases the risk of pipe blockage, and reduces processing capacity. Additionally, when the first thermal decomposition liquid is obtained by cooling the thermally decomposed vapor generated during the first thermal decomposition step, these deposits may reduce the cooling efficiency.

### <Quantification Method of Deposit-Inducing Substances in First Thermal Decomposition Liquid>

In the present example and comparative examples, the deposit-inducing substances in the first thermal decomposition liquid were quantified according to the following procedure.

The obtained first thermal decomposition liquid from the thermal decomposition experiment of the styrene-based resin composition was heated for 30 minutes so that the internal temperature of the first thermal decomposition liquid reached 60° C. Then, 10 g of the heated first thermal decomposition liquid was extracted and weighed, followed by cooling in a refrigerator at 0° C. for 1 hour. The cooled liquid was subjected to vacuum filtration using a PTFE-made membrane filter (T100A047A) manufactured by Toyo Roshi Kaisha, Ltd. The collected filtrate was then washed with 1 mL of MEK cooled to 0° C. Next, the washed filtrate was dried in a vacuum dryer at 60° C. for 30 minutes, and the dry weight of the filtrate was measured. The mass concentration of the deposit-inducing substances was determined from the ratio of the dry weight of the filtrate to the 10 g of the first thermal decomposition liquid.

Through the analysis on deposits on the inner wall surface of the heat exchange (commonly referred to as the surface of the cooling tube), a consistent correlation was confirmed between the components contained in the first thermal decomposition liquid (i.e., deposit-inducing substances) and the formation of deposits.

When the amount of deposit-inducing substances in the first thermal decomposition liquid obtained in the first thermal decomposition step was 0 to less than 0.2 wt %, no deposits were observed in the liquefaction apparatus used to cool the thermally decomposed vapor immediately after the production of the first thermal decomposition liquid. On the other hand, when the amount of deposit-inducing substances was 0.2 wt % or more and less than 0.45 wt %, thin film-like deposits were observed on the inner wall surface of the heat exchange of the liquefaction apparatus immediately after the production of the first thermal decomposition liquid. Furthermore, when the amount of deposit-inducing substances in the first thermal decomposition liquid was 0.45 wt % or higher, deposits were apparently observed.

Additionally, in the first thermal decomposition step, the step of producing the first thermal decomposition liquid by cooling the thermally decomposed vapor after heating the fluid was continuously operated for one week without disassembly and cleaning, and the presence of deposits on the inner wall surface of the heat exchange of the liquefaction apparatus was evaluated. Samples with thick deposits confirmed on the surface were rated as “Poor” (e.g., the state in FIG. 8), samples with thin film-like deposits in a small amount were rated as “Fair” (e.g., the state in FIG. 7), and samples with no observable deposits were rated as “Excellent” (e.g., the state in FIG. 6). These evaluations were recorded in Tables 1-2 to 1-5 under the category of the presence of deposits (after one-week operation).

When the first thermal decomposition step was continuously operated for one week without disassembly and cleaning, no deposits were observed in the liquefaction apparatus when the amount of deposit-inducing substances in the first thermal decomposition liquid was 0 to less than 0.15 wt %. However, when the amount of deposit-inducing substances was 0.15 wt % or more and less than 0.35 wt %, thin film-like deposits were observed on the inner wall surface of the heat exchange of the liquefaction apparatus in the step of producing the first thermal decomposition liquid. Additionally, when the amount of deposit-inducing substances in the first thermal decomposition liquid was 0.35 wt % or higher, deposits were apparently observed.

The materials used in Examples and Comparative Examples are as follows.

### <GPPS>

GPPS 680 manufactured by PS Japan Corporation was used.

### <Post-Consumer Material: Polystyrene Material A>

A styrene-based resin composition recovered from home appliances was used. The weight-average molecular weight (Mw) was 110,000.

### <Recovered and Sorted Material: Polystyrene Material B>

A styrene-based resin composition sorted as polystyrene from waste plastics collected under the Japanese Law for Promotion of Sorted Collection and Recycling of Containers and Packaging was used. The weight-average molecular weight (Mw) was 110,000.

### <Recovered and Sorted Material: Polystyrene Material C>

A styrene-based resin composition sorted as polystyrene from market-collected waste plastics from waste plastics was used. The weight-average molecular weight (Mw) was 100,000.

### <Recovered and Sorted Material: Polystyrene Material D>

A styrene-based resin composition sorted as polystyrene from market-collected waste plastics was used. The weight-average molecular weight (Mw) was 105,000.

### <Polyethylene>

SGF4960 manufactured by Braskem S. A. was used.

### <Polypropylene>

MA3 manufactured by Japan Polypropylene Corporation was used.

### Example 1

A mixed liquid was prepared by adding toluene to GPPS (99 parts by mass) and polyethylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (1). The devolatilized and collected fluid (1) was subjected to thermal decomposition under the conditions of 450° C. and 34 hPa, and the gas containing the styrene monomer generated by the thermal decomposition was condensed with a cooling tube cooled at −10° C. to obtain a first thermal decomposition liquid (1) containing the styrene monomer.

The crude residue rate (B) in the vessel used for thermal decomposition, the weight loss rate (C) and weight loss rate (D), and the residue rate (A) calculated from these values are listed in Table 1-2.

### Example 2

A mixed liquid was prepared by adding toluene to GPPS (99 parts by mass) and polypropylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (2). The devolatilized and collected fluid (2) was subjected to thermal decomposition under the conditions of 450° C. and 34 hPa, and the gas generated by the thermal decomposition was condensed with a cooling tube cooled at −10° C. to obtain a first thermal decomposition liquid (2).

The crude residue rate (B) in the vessel used for thermal decomposition, the weight loss rate (C) and (D), and the residue rate (A) calculated from these values are listed in Table 1-2.

### Example 3

A mixed liquid was prepared by adding toluene to GPPS (99 parts by mass) and talc (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (3). The devolatilized and collected fluid (3) was subjected to thermal decomposition under the conditions of 450° C. and 34 hPa, and the gas generated by the thermal decomposition was condensed with a cooling tube cooled at −10° C. to obtain a first thermal decomposition liquid (3).

The crude residue rate (B) in the vessel used for thermal decomposition, the weight loss rate (C) and (D), and the residue rate (A) calculated from these values are listed in Table 1-2.

### Example 4

A mixed liquid was prepared by adding methyl ethyl ketone to GPPS (99 parts by mass) and talc (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (4). The devolatilized and collected fluid (4) was subjected to thermal decomposition under the conditions of 450° C. and 34 hPa, and the gas generated by the thermal decomposition was condensed with a cooling tube cooled at −10° C. to obtain a first thermal decomposition liquid (4). The crude residue rate (B) in the vessel used for thermal decomposition, the weight loss rates (C) and (D), and the residue rate (A) calculated from these values are listed in Table 1-2.

### Example 5

A mixed liquid was prepared by adding toluene to the polystyrene material A (100 parts by mass) to achieve a 10 mass % concentration (solid content concentration), followed by stirring. The slurry was purified by suction filtration using a Kiriyama funnel and Kiriyama filter paper 5B. The purified slurry was then devolatilized using a vacuum dryer under conditions of 160° C. and 40 hPa to obtain a fluid (5). The devolatilized and collected fluid (4) was subjected to thermal decomposition under the conditions of 450° C. and 34 hPa, and the gas generated by the thermal decomposition was condensed with a cooling tube cooled at −10° C. to obtain a first thermal decomposition liquid (5). The crude residue rate (B) in the container used for decomposition, the weight loss rates (C) and (D) analyzed by TGA analysis, and the residue rate (A) calculated from these values are listed in Table 1-2.

### Example 6

A mixed liquid was prepared by adding toluene to the polystyrene material B (100 parts by mass) to achieve a 10 mass % concentration, followed by stirring. The slurry was purified by suction filtration using a Kiriyama funnel and Kiriyama filter paper 5B. The purified slurry was then devolatilized using a vacuum dryer under conditions of 160° C. and 40 hPa to obtain a fluid (6). The devolatilized and collected fluid (6) was subjected to thermal decomposition under the conditions of 450° C. and 34 hPa, and the gas containing the styrene monomer generated by the thermal decomposition was condensed with a cooling tube cooled at −10° C. to obtain a first thermal decomposition liquid (6). The crude residue rate (B) in the container used for decomposition, the weight loss rates (C) and (D), and the residue rate (A) calculated from these values are listed in Table 1-2.

### Example 7

A mixed liquid was prepared by adding MEK to the polystyrene material B (100 parts by mass) to achieve a 10 mass % concentration, followed by stirring. The slurry was purified by suction filtration using a Kiriyama funnel and Kiriyama filter paper 5B. The purified slurry was then devolatilized using a vacuum dryer under conditions of 160° C. and 40 hPa to obtain a fluid (7). The devolatilized and collected fluid (7) was subjected to thermal decomposition under the conditions of 450° C. and 34 hPa, and the gas generated by the thermal decomposition was condensed with a cooling tube cooled at −10° C. to obtain a first thermal decomposition liquid (7). The crude residue rate (B) in the container used for decomposition, the weight loss rates (C) and (D), and the residue rate (A) calculated from these values are listed in Table 1-2.

### Example 8

A mixed liquid was prepared by adding toluene to the polystyrene material B (100 parts by mass) to achieve a 10 concentration, followed by stirring, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (8). The devolatilized and collected fluid (8) was subjected to thermal decomposition under the conditions of 450° C. and 34 hPa, and the gas generated by the thermal decomposition was condensed with a cooling tube cooled at −10° C. to obtain a first thermal decomposition liquid (8). The crude residue rate (B) in the container used for decomposition, the weight loss rates (C) and (D), and the residue rate (A) calculated from these values are listed in Table 1-2.

### Example 9

A mixed liquid was prepared by adding MEK to the polystyrene material B (100 parts by mass) and performing stirring to achieve a 10 mass % concentration, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (9). The devolatilized and collected fluid (9) was subjected to thermal decomposition under the conditions of 450° C. and 34 hPa, and the gas generated by the thermal decomposition was condensed with a cooling tube cooled at −10° C. to obtain a first thermal decomposition liquid (9). The crude residue rate (B) in the container used for decomposition, the weight loss rates (C) and (D), and the residue rate (A) calculated from these values are listed in Table 1-2.

### Example 10

A mixed liquid was prepared by adding ethylbenzene to GPPS (99 parts by mass) and polyethylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (10). The devolatilized and collected fluid (10) was subjected to thermal decomposition under the conditions of 450° C. and 34 hPa, and the gas containing the styrene monomer generated by the thermal decomposition was condensed with a cooling tube cooled at −10° C. to obtain a first thermal decomposition liquid (10) containing the styrene monomer.

The crude residue rate (B) in the vessel used for thermal decomposition, the weight loss rates (C) and (D), and the residue rate (A) calculated from these values are listed in Table 1-3.

### Example 11

A mixed liquid was prepared by adding ethylbenzene to GPPS (99 parts by mass) and polypropylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (11). The devolatilized and collected fluid (11) was subjected to thermal decomposition under the conditions of 450° C. and 34 hPa, and the gas generated by the thermal decomposition was condensed with a cooling tube cooled at −10° C. to obtain a first thermal decomposition liquid (11).

The crude residue rate (B) in the vessel used for thermal decomposition, the weight loss rates (C) and (D), and the residue rate (A) calculated from these values are listed in Table 1-3.

### Example 12

A mixed liquid was prepared by adding ethylbenzene to GPPS (99 parts by mass) and talc (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (12). The devolatilized and collected fluid (12) was subjected to thermal decomposition under the conditions of 450° C. and 34 hPa, and the gas generated by the thermal decomposition was condensed with a cooling tube cooled at −10° C. to obtain a first thermal decomposition liquid (12).

The crude residue rate (B) in the vessel used for thermal decomposition, the weight loss rates (C) and (D), and the residue rate (A) calculated from these values are listed in Table 1-3.

### Example 13

A mixed liquid was prepared by adding toluene to the polystyrene material A (100 parts by mass) to achieve a 10 mass % concentration (solid content concentration), followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (13). The devolatilized and collected fluid (13) was subjected to thermal decomposition under the conditions of 450° C. and 34 hPa, and the gas generated by the thermal decomposition was condensed with a cooling tube cooled at −10° C. to obtain a first thermal decomposition liquid (13). The crude residue rate (B) in the container used for decomposition, the weight loss rates (C) and (D) analyzed by TGA analysis, and the residue rate (A) calculated from these values are listed in Table 1-3.

### Example 14

A mixed liquid was prepared by adding ethylbenzene to the polystyrene material B (100 parts by mass) to achieve a 10 mass % concentration, followed by stirring. The mixed liquid was purified by suction filtration using a Kiriyama funnel and Kiriyama filter paper 5B. The purified slurry was then devolatilized using a vacuum dryer under conditions of 160° C. and 40 hPa to obtain a fluid (14). The devolatilized and collected fluid (14) was subjected to thermal decomposition under the conditions of 450° C. and 34 hPa, and the gas generated by the thermal decomposition was condensed with a cooling tube cooled at −10° C. to obtain a first thermal decomposition liquid (14). The crude residue rate (B) in the container used for decomposition, the weight loss rates (C) and (D), and the residue rate (A) calculated from these values are listed in Table 1-3.

### Example 15

A mixed liquid was prepared by adding ethylbenzene to the polystyrene material B (100 parts by mass) to achieve a 10 mass % concentration, followed by stirring, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (15). The devolatilized and collected fluid (15) was subjected to thermal decomposition under the conditions of 450° C. and 34 hPa, and the gas generated by the thermal decomposition was condensed with a cooling tube cooled at −10° C. to obtain a first thermal decomposition liquid (15). The crude residue rate (B) in the container used for decomposition, the weight loss rates (C) and (D), and the residue rate (A) calculated from these values are listed in Table 1-3.

### Example 16

A mixed liquid was prepared by adding ethylbenzene to the polystyrene material C (100 parts by mass) to achieve a 10 mass % concentration, followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (16). The devolatilized and collected fluid (16) was subjected to thermal decomposition under the conditions of 450° C. and 34 hPa, and the gas generated by the thermal decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-4.

### Example 17

A mixed liquid was prepared by adding ethylbenzene to the polystyrene material C (100 parts by mass) to achieve a 10 mass % concentration, followed by stirring. Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (17). The devolatilized and collected fluid (17) was subjected to thermal decomposition under the conditions of 450° C. and 34 hPa, and the gas generated by the thermal decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-4.

### Example 18

A mixed liquid was prepared by adding ethylbenzene to the polystyrene material D (100 parts by mass) to achieve a 10 mass % concentration, followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (18). The devolatilized and collected fluid (18) was subjected to thermal decomposition under the conditions of 450° C. and 34 hPa, and the gas generated by the thermal decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-4.

### Example 19

A mixed liquid was prepared by adding ethylbenzene to the polystyrene material D (100 parts by mass) to achieve a 10 mass % concentration, followed by stirring. Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (19). The devolatilized and collected fluid (19) was subjected to thermal decomposition under the conditions of 450° C. and 34 hPa, and the gas generated by the thermal decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-4.

### Example 20

A mixed liquid was prepared by adding styrene monomer to GPPS (99 parts by mass) and polyethylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (20). The devolatilized and collected fluid (20) was subjected to thermal decomposition under the conditions of 450° C. and 34 hPa, and the gas containing the styrene monomer generated by the thermal decomposition was condensed with a cooling tube cooled at −10° C. to obtain a first thermal decomposition liquid (20) containing the styrene monomer.

The crude residue rate (B) in the vessel used for thermal decomposition, the weight loss rates (C) and (D), and the residue rate (A) calculated from these values are listed in Table 1-4.

### Example 21

A mixed liquid was prepared by adding styrene monomer to GPPS (99 parts by mass) and polypropylene (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (21). The devolatilized and collected fluid (21) was subjected to thermal decomposition under the conditions of 450° C. and 34 hPa, and the gas generated by the thermal decomposition was condensed with a cooling tube cooled at −10° C. to obtain a first thermal decomposition liquid (21).

The crude residue rate (B) in the vessel used for thermal decomposition, the weight loss rates (C) and (D), and the residue rate (A) calculated from these values are listed in Table 1-4.

### Example 22

A mixed liquid was prepared by adding styrene monomer to GPPS (99 parts by mass) and talc (1 part by mass) to achieve a 10 mass % concentration (solid content concentration), followed by stirring. Then, the mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (22). The devolatilized and collected fluid (22) was subjected to thermal decomposition under the conditions of 450° C. and 34 hPa, and the gas generated by the thermal decomposition was condensed with a cooling tube cooled at −10° C. to obtain a first thermal decomposition liquid (22).

The crude residue rate (B) in the vessel used for thermal decomposition, the weight loss rates (C) and (D), and the residue rate (A) calculated from these values are listed in Table 1-4.

### Example 23

A mixed liquid was prepared by adding styrene monomer to the polystyrene material A (100 parts by mass) to achieve a 10 mass % concentration (solid content concentration), followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (23). The devolatilized and collected fluid (23) was subjected to thermal decomposition under the conditions of 450° C. and 34 hPa, and the gas generated by the first thermal decomposition (23) was condensed with a cooling tube cooled at −10° C. to obtain a first thermal decomposition liquid (23). The crude residue rate (B) in the container used for decomposition, the weight loss rates (C) and (D) analyzed by TGA analysis, and the residue rate (A) calculated from these values are listed in Table 1-4.

### Example 24

A mixed liquid was prepared by adding styrene monomer to the polystyrene material B (100 parts by mass) to achieve a 10 mass % concentration, followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (24). The devolatilized and collected fluid (24) was subjected to thermal decomposition under the conditions of 450° C. and 34 hPa, and the gas generated by the thermal decomposition was condensed with a cooling tube cooled at −10° C. to obtain a first thermal decomposition liquid (24). The crude residue rate (B) in the container used for decomposition, the weight loss rates (C) and (D), and the residue rate (A) calculated from these values are listed in Table 1-4.

### Example 25

A mixed liquid was prepared by adding styrene monomer to the polystyrene material B (100 parts by mass) to achieve a 10 mass % concentration, followed by stirring, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (25). The devolatilized and collected fluid (25) was subjected to thermal decomposition under the conditions of 450° C. and 34 hPa, and the gas generated by the thermal decomposition was condensed with a cooling tube cooled at −10° C. to obtain a first thermal decomposition liquid (25). The crude residue rate (B) in the container used for decomposition, the weight loss rates (C) and (D), and the residue rate (A) calculated from these values are listed in Table 1-4.

### Example 26

A mixed liquid was prepared by adding styrene monomer to the polystyrene material C (100 parts by mass) to achieve a 10 mass % concentration, followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (26). The devolatilized and collected fluid (26) was subjected to thermal decomposition under the conditions of 450° C. and 34 hPa, and the gas generated by the thermal decomposition was condensed with a cooling tube cooled at −10° C. to obtain a first thermal decomposition liquid (26). The crude residue rate (B) in the container used for decomposition, the weight loss rates (C) and (D), and the residue rate (A) calculated from these values are listed in Table 1-4.

### Example 27

A mixed liquid was prepared by adding styrene monomer to polystyrene material C (100 parts by mass) to achieve a 10 mass % concentration, followed by stirring, and Radiolite #100 (diatomaceous earth) manufactured by Showa Chemical Industry Co., Ltd. was added at 4 mass % relative to the weight of the slurry and stirred to prepare a diatomaceous earth-containing mixture. The diatomaceous earth-containing mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (27). The devolatilized and collected fluid (27) was subjected to thermal decomposition under the conditions of 450° C. and 34 hPa, and the gas generated by the thermal decomposition was condensed with a cooling tube cooled at −10° C. to obtain a first thermal decomposition liquid (27). The crude residue rate (B) in the container used for decomposition, the weight loss rates (C) and (D), and the residue rate (A) calculated from these values are listed in Table 1-4.

### Example 28

A mixed liquid was prepared by adding styrene monomer to the polystyrene material D (100 parts by mass) to achieve a 10 mass % concentration, followed by stirring. The mixed liquid was purified by suction filtration using a Kiriyama funnel and Kiriyama filter paper 5B. Subsequently, the purified mixed liquid was subjected to a devolatilization process using a vacuum dryer under conditions of 160° C. and 40 hPa to obtain a fluid (28). The devolatilized and collected fluid (28) was subjected to thermal decomposition under the conditions of 450° C. and 34 hPa, and the gas generated by the thermal decomposition was condensed with a cooling tube cooled at −10° C. to obtain a first thermal decomposition liquid (28). The crude residue rate (B) in the container used for decomposition, the weight loss rates (C) and (D), and the residue rate (A) calculated from these values are listed in Table 1-4.

### Example 29

A mixed liquid was prepared by adding styrene monomer to the polystyrene material D (100 parts by mass) to achieve a 10 mass % concentration, followed by stirring, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by suction filtration using a Kiriyama funnel and Kiriyama filter paper 5B. Subsequently, the purified diatomaceous earth-containing mixed liquid was then devolatilized using a vacuum dryer under conditions of 160° C. and 40 hPa to obtain a fluid (29). The devolatilized and collected fluid (29) was subjected to thermal decomposition under the conditions of 450° C. and 34 hPa, and the gas generated by the thermal decomposition was condensed with a cooling tube cooled at −10° C. to obtain a first thermal decomposition liquid (29). The crude residue rate (B) in the container used for decomposition, the weight loss rates (C) and (D), and the residue rate (A) calculated from these values are listed in Table 1-4.

### Example 30

A mixed liquid was prepared by adding styrene monomer to the polystyrene material B (100 parts by mass) to achieve a 10 mass % concentration, followed by stirring, and Radiolite #100 (diatomaceous earth) manufactured by Showa Chemical Industry Co., Ltd. was added at 4 mass % relative to the weight of the slurry and stirred to prepare a diatomaceous earth-containing mixture. The diatomaceous earth-containing mixed liquid was purified by pressure filtration using a pressure filter and a 2/2 twill weave nylon filter cloth with an air permeability of 1000 cm³/cm²·min, under nitrogen at 0.2 MPa. Subsequently, the purified diatomaceous earth-containing mixture was devolatilized under conditions of 160° C. and 40 hPa to obtain a fluid (30). Using the thermal decomposition apparatus illustrated in FIG. 3, the devolatilized and collected fluid (30) was subjected to thermal decomposition under conditions of 450° C. and 34 hPa, and the gas generated by the thermal decomposition was condensed with a cooling tube cooled at −10° C. to obtain a first thermal decomposition liquid (30). The crude residue rate (B) in the apparatus used for decomposition, the weight loss rates (C) and (D), and the residue rate (A) calculated from these values were 0.35%, and the content of deposit-inducing substances in the first thermal decomposition liquid was 0.02 wt %.

Then, the first thermal decomposition liquid (30) was distilled under conditions of 60° C. and 53 Torr in the first distillation step, and the obtained second fraction was distilled under conditions of 100° C. and 22 Torr in the second distillation step to obtain the styrene monomer as the third fraction.

The total yield of styrene monomer recovered by distillation in the above steps was 55%.

### Example 31

A mixed liquid was prepared by adding ethylbenzene to the polystyrene material D (100 parts by mass) to achieve a 10 mass % concentration, followed by stirring. The mixture was then purified by pressure filtration using a pressure filter and a 2/2 twill weave nylon filter cloth with an air permeability of 1000 cm³/cm²·min, under nitrogen at 0.2 MPa. Subsequently, the purified mixed liquid was devolatilized under conditions of 160° C. and 40 hPa to obtain a fluid (31). Using the thermal decomposition apparatus illustrated in FIG. 3, the devolatilized and collected fluid (31) was subjected to thermal decomposition under conditions of 450° C. and 34 hPa, and the gas generated by the thermal decomposition was condensed with a cooling tube cooled at −10° C. to obtain a first thermal decomposition liquid (31). The crude residue rate (B) in the apparatus used for decomposition, the weight loss rates (C) and (D), and the residue rate (A) calculated from these values were 0.34%, and the content of deposit-inducing substances in the first thermal decomposition liquid was 0.02 wt %.

### Example 32

A mixed liquid was prepared by adding toluene to GPPS (99 parts by mass) and polyethylene (1 part by mass) to achieve a 10 mass % concentration (solid content concentration), followed by stirring. Then, the mixed liquid was purified by pressure filtration using a pressure filter and a 2/2 twill weave nylon filter cloth with an air permeability of 1000 cm³/cm²·min, under nitrogen at 0.2 MPa. Subsequently, the purified mixed liquid was devolatilized under conditions of 160° C. and 40 hPa to obtain a fluid (32). Using the thermal decomposition apparatus illustrated in FIG. 3, the devolatilized and collected fluid (32) was subjected to thermal decomposition under conditions of 450° C. and 34 hPa, and the gas generated by the thermal decomposition was condensed with a cooling tube cooled at −10° C. to obtain a first thermal decomposition liquid (32). The crude residue rate (B) in the apparatus used for decomposition, the weight loss rates (C) and (D), and the residue rate (A) calculated from these values were 0.05%, and the content of deposit-inducing substances in the first thermal decomposition liquid was 0.01 wt %.

### Example 33

A mixed liquid was prepared by adding ethylbenzene to the polystyrene material B (100 parts by mass) to achieve a 10 mass % concentration, followed by stirring, and Radiolite #100 (diatomaceous earth) manufactured by Showa Chemical Industry Co., Ltd. was added in an amount of 4 mass % relative to the weight of the mixed liquid and stirred to prepare a diatomaceous earth-containing mixed liquid. The diatomaceous earth-containing mixed liquid was purified by pressure filtration using a pressure filter and a 2/2 twill weave nylon filter cloth with an air permeability of 1000 cm³/cm²·min, under nitrogen at 0.2 MPa. Subsequently, the purified diatomaceous earth-containing mixture was devolatilized under conditions of 160° C. and 40 hPa to obtain a fluid (33). Then, using the thermal decomposition apparatus illustrated in FIG. 3, the devolatilized and collected fluid (33) was subjected to thermal decomposition under conditions of 450° C. and 34 hPa, and the gas generated by the thermal decomposition was condensed with a cooling tube cooled at −10° C. to obtain a first thermal decomposition liquid (33).

Then, the first thermal decomposition liquid (33) was distilled under conditions of 60° C. and 53 Torr in the first distillation step, and the obtained second fraction was distilled under conditions of 100° C. and 22 Torr in the second distillation step to obtain the styrene monomer as the third fraction. Meanwhile, the second thermal decomposition step was performed on the fourth fraction, which was not included in the third fraction. Specifically, a SUS-made reaction vessel filled with the fourth fraction was placed inside a casting heater, a SUS-made cover with a branch pipe was attached, and the bolts were tightened with a wrench. A dedicated adapter with an O-ring was attached to the branch pipe, and a condenser, a distillation adapter with a branch for a vacuum hose connection, and a thermal decomposition liquid collection vessel were each attached, with silicone grease applied. Then, a three-way cock was attached on the branch for the vacuum hose connection, and a nitrogen balloon and a vacuum pump were connected. Additionally, the temperature was monitored using thermocouples installed inside the casting heater and the container.

The second thermal decomposition step was performed at 450° C. under decompressed pressure by setting the vacuum pump to 34 hPa while feeding the coolant at −10° C. and adjusting the output to the cast-in heater. The thermally decomposed vapor was liquefied by the condenser through which the coolant flowed, and the second thermal decomposition liquid was collected in the collection vessel. The obtained second thermal decomposition liquid was distilled using a distillation apparatus at 13 hPa, with the temperature increased from 50° C. to 80° C. in increments of 5° C. every 5 minutes, thereby removing the light-boiling fraction (fifth fraction). Meanwhile, the heavy-boiling fraction (sixth fraction) was discharged to the outside. The light-boiling fraction (fifth fraction) was recycled by adding it to the first thermal decomposition liquid as needed.

The crude residue rate (B) in the apparatus used for decomposition, the weight loss rates (C) and (D), and the residue rate (A) calculated from these values were 0.05%, and the content of deposit-inducing substances in the first thermal decomposition liquid was 0.01 wt %.

The total yield of styrene monomer recovered by distillation in the above steps was 70%.

### Comparative Example 1

A mixture of GPPS (99 parts by mass) and polyethylene (1 part by mass) was prepared, and was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 2

A mixture of GPPS (99 parts by mass) and polypropylene (1 part by mass) was prepared, and was subjected to thermal decomposition under conditions of 450° C. and 34 hPa without being dissolved in a solvent and without undergoing purification and devolatilization steps. The gas containing the styrene monomer generated by the thermal decomposition was condensed with a cooling tube cooled at −10° C. to obtain a thermal decomposition liquid. The crude residue rate (B) in the container used for decomposition, the weight loss rates (C) and (D), and the residue rate (A) calculated from these values are listed in Table 1-5.

### Comparative Example 3

GPPS (99 parts by mass) and talc (1 part by mass) ware mixed, and were subjected to thermal decomposition under conditions of 450° C. and 34 hPa without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 4

The polystyrene material A (100 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the analyzed weight loss rates (C) and (D), and the residue rate (A) calculated from these values are listed in Table 1-5.

### Comparative Example 5

The production of styrene monomer was carried out using an apparatus in which the thermal decomposition apparatus 10 and liquefaction apparatus L in FIG. 3 were connected, similar to the production apparatus for styrene monomer used in Examples 30 to 33. The polystyrene material B (100 parts by mass) was subjected to thermal decomposition under conditions of 450° C. and 34 hPa without being dissolved in a solvent and without undergoing purification and devolatilization steps. The gas generated by the decomposition was condensed using a liquefaction apparatus cooled at −10° C. to obtain a decomposed liquid. The crude residue rate (B) in the container used for decomposition, the weight loss rates (C) and (D), and the residue rate (A) calculated from these values are listed in Table 1-5.

The connecting section C1P, i.e., the glass tube, immediately before the production of styrene monomer in Comparative Example 5, is indicated in FIG. 6. The connecting section C1P, i.e., the glass tube, during the production of styrene monomer, is indicated in FIG. 7. Furthermore, the connecting section C1P, i.e., the glass tube, immediately after the production of styrene monomer, is indicated in FIG. 8. FIGS. 6 to 8 are photographs indicating the condition of the glass tube over time. It was confirmed from these photographs that the initially transparent glass tube gradually became clouded due to a thin film-like, milky-white deposit, and ultimately, a black substance Res adhered to the entire inner wall of the connecting section C1P and the liquefaction apparatus L, causing almost complete blockage.

### Comparative Example 6

A mixture of GPPS (90 parts by mass) and the polystyrene material B (10 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 7

A mixture of GPPS (75 parts by mass) and the polystyrene material B (25 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 8

A mixture of GPPS (50 parts by mass) and the polystyrene material B (50 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 9

A mixture of GPPS (30 parts by mass) and the polystyrene material B (70 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-5.

### Comparative Example 10

A mixture of GPPS (15 parts by mass) and the polystyrene material B (85 parts by mass) was subjected to thermal decomposition under conditions of 450° C. and 34 hPa without being dissolved in a solvent and without undergoing purification and devolatilization steps. The gas generated by the decomposition was condensed with a cooling tube cooled at −10° C. to obtain a decomposed liquid. The crude residue rate (B) in the container used for decomposition, the weight loss rates (C) and (D), and the residue rate (A) calculated from these values are listed in Table 1-2.

TABLE 1-1

| | GPPS | Suitability to |
|---|---|---|
| Solvent | (mass %) | prepare slurry |
| Toluene | 5 | Good |
| Toluene | 10 | Good |
| Toluene | 20 | Good |
| Toluene | 30 | Good |
| Toluene | 40 | Good |
| Ethylbenzene | 10 | Good |
| Ethylbenzene | 20 | Good |
| Ethylbenzene | 30 | Good |
| Ethylbenzene | 40 | Good |
| Styrene | 10 | Good |
| Styrene | 20 | Good |
| Styrene | 30 | Good |
| Styrene | 40 | Good |
| Methyl ethyl ketone | 10 | Good |
| Methyl ethyl ketone | 20 | Good |
| Methyl ethyl ketone | 30 | Good |
| Methyl ethyl ketone | 40 | Good |
| Tetrahydrofuran | 10 | Good |
| MeOH | 10 | Bad |

TABLE 1-2

| | Example 1 | Example 2 | Example 3 | Example 4 | Example 5 | Example 6 | Example 7 | Example 8 | Example 9 |
|---|---|---|---|---|---|---|---|---|---|
| Solvent | Toluene | Toluene | Toluene | MEK | Toluene | Toluene | MEK | Toluene | MEK |
| Secondary solvent | — | — | — | — | — | — | — | — | — |
| Residue rate A (%) | 0.00 | 0.00 | 0.05 | 0.06 | 0.12 | 0.34 | 0.06 | 0.32 | 0.14 |
| Crude residue rate B (%) | 0 | 0 | 0.1 | 0.1 | 0.24 | 0.43 | 0.09 | 0.4 | 0.2 |
| Weight loss rate C (%) | 0 | 0 | 49.07 | 58.25 | 49.07 | 78.84 | 69.56 | 79 | 70 |
| Weight loss rate D (%) | 0 | 0 | 7.11 | 15.4 | 7.11 | 14.2 | 10.55 | 14 | 10 |
| Presence of deposits on inner | Excellent | Excellent | Excellent | Excellent | Excellent | Excellent | Excellent | Excellent | Excellent |
| wall of cooling tube or heat | | | | | | | | | |
| exchanger (immediately after | | | | | | | | | |
| thermal decomposition step) | | | | | | | | | |
| Adhesion to inner wall of | Excellent | Excellent | Excellent | Excellent | Excellent | Excellent | Excellent | Excellent | Excellent |
| cooling tube or heat exchanger | | | | | | | | | |
| (after 1-week operation) | | | | | | | | | |
| Content of deposit-inducing | <0.01 | <0.01 | <0.01 | <0.01 | 0.01 | 0.02 | 0.01 | 0.01 | 0.01 |
| substances in first themally | | | | | | | | | |
| decomposed liquid (wt %) | | | | | | | | | |

TABLE 1-3

| | Example | Example | Example | Example | Example | Example | Example | Example | Example | Example |
|---|---|---|---|---|---|---|---|---|---|---|
| | 10 | 11 | 12 | 13 | 14 | 15 | 16 | 17 | 18 | 19 |
| Solvent | EB | EB | EB | EB | EB | EB | EB | EB | EB | EB |
| Secondary solvent | — | — | — | — | — | — | — | — | — | — |
| Residue rate A (%) | 0.00 | 0.00 | 0.07 | 0.12 | 0.34 | 0.32 | 0.31 | 0.26 | 0.23 | 0.20 |
| Crude residue rate B (%) | 0 | 0 | 0.15 | 0.25 | 0.43 | 0.4 | 0.39 | 0.36 | 0.3 | 0.28 |
| Weight loss rate C (%) | 0 | 0 | 49.1 | 48.9 | 78.84 | 78.84 | 78.7 | 71 | 77 | 70 |
| Weight loss rate D (%) | 0 | 0 | 7.2 | 7.3 | 14.2 | 14.2 | 14.2 | 14 | 13.5 | 12 |
| Presence of deposits on inner | Excellent | Excellent | Excellent | Excellent | Excellent | Excellent | Excellent | Excellent | Excellent | Excellent |
| wall of cooling tube or heat | | | | | | | | | | |
| exchanger (immediately after | | | | | | | | | | |
| thermal decomposition step) | | | | | | | | | | |
| Adhesion to inner wall of | Excellent | Excellent | Excellent | Excellent | Excellent | Excellent | Excellent | Excellent | Excellent | Excellent |
| cooling tube or heat exchanger | | | | | | | | | | |
| (after 1-week operation) | | | | | | | | | | |
| Content of deposit-inducing | < 0.01 | <0.01 | < 0.01 | < 0.01 | 0.01 | <0.01 | 0.18 | 0.14 | 0.17 | 0.13 |
| substances in first themally | | | | | | | | | | |
| decomposed liquid (wt %) | | | | | | | | | | |

TABLE 1-4

| | Example | Example | Example | Example | Example | Example | Example | Example | Example | Example |
|---|---|---|---|---|---|---|---|---|---|---|
| | 20 | 21 | 22 | 23 | 24 | 25 | 26 | 27 | 28 | 29 |
| Solvent | SM | SM | SM | SM | SM | SM | SM | SM | SM | SM |
| Secondary solvent | — | — | — | — | — | — | | — | — | — |
| Residue rate A (%) | 0.00 | 0.00 | 0.08 | 0.13 | 0.36 | 0.32 | 0.32 | 0.26 | 0.24 | 0.20 |
| Crude residue rate B (%) | 0 | 0 | 0.16 | 0.26 | 0.45 | 0.41 | 0.4 | 0.37 | 0.31 | 0.28 |
| Weight loss rate C (%) | 0 | 0 | 49 | 49 | 79 | 78 | 79 | 71 | 76.9 | 70.5 |
| Weight loss rate D (%) | 0 | 0 | 7 | 7.1 | 14 | 14 | 14.1 | 14.5 | 13.5 | 11.5 |
| Presence of deposits on inner | Excellent | Excellent | Excellent | Excellent | Excellent | Excellent | Excellent | Excellent | Excellent | Excellent |
| wall of cooling tube or heat | | | | | | | | | | |
| exchanger (immediately after | | | | | | | | | | |
| thermal decomposition step) | | | | | | | | | | |
| Adhesion to inner wall of | Excellent | Excellent | Excellent | Excellent | Excellent | Excellent | Excellent | Excellent | Excellent | Excellent |
| cooling tube or heat exchanger | | | | | | | | | | |
| (after 1-week operation) | | | | | | | | | | |
| Content of deposit-inducing | <0.01 | <0.01 | 0.01 | 0.01 | 0.01 | <0.01 | 0.19 | 0.15 | 0.14 | 0.13 |
| substances in first themally | | | | | | | | | | |
| decomposed liquid (wt %) | | | | | | | | | | |

TABLE 1-5

| | Comp. | Comp. | Comp. | Comp. | Comp. | Comp. | Comp. | Comp. | Comp. | Comp. |
|---|---|---|---|---|---|---|---|---|---|---|
| | Ex. 1 | Ex. 2 | Ex. 3 | Ex. 4 | Ex. 5 | Ex. 6 | Ex. 7 | Ex. 8 | Ex. 9 | Ex. 10 |
| Solvent | — | — | — | — | — | — | — | — | — | — |
| Secondary solvent | — | — | — | — | — | — | — | — | — | — |
| Residue rate A (%) | 0.00 | 0.00 | 0.80 | 0.83 | 1.88 | 0.42 | 0.61 | 0.79 | 1.18 | 1.52 |
| Crude residue rate B (%) | 0.09 | 0.12 | 1 | 1.03 | 2.52 | 0.6 | 0.85 | 1.09 | 1.61 | 2.05 |
| Weight loss rate C (%) | 0.86 | 0.88 | 79.91 | 80.89 | 74.71 | 70.6 | 72 | 72.5 | 73 | 74.1 |
| Weight loss rate D (%) | 1 | 1 | 44.6 | 45.31 | 35.36 | 33.2 | 33.5 | 34.1 | 34.8 | 35 |
| Presence of deposits on inner | Poor | Poor | Fair | Poor | Poor | Poor | Poor | Poor | Poor | Poor |
| wall of cooling tube or heat | | | | | | | | | | |
| exchanger (immediately after | | | | | | | | | | |
| thermal decomposition step) | | | | | | | | | | |
| Adhesion to inner wall of | Poor | Poor | Fair | Poor | Poor | Poor | Poor | Poor | Poor | Poor |
| cooling tube or heat exchanger | | | | | | | | | | |
| (after 1-week operation) | | | | | | | | | | |
| Content of deposit-inducing | 0.5 | 0.5 | 0.2 | 0.6 | 0.98 | 0.45 | 0.51 | 0.69 | 0.76 | 0.88 |
| substances in first themally | | | | | | | | | | |
| decomposed liquid (wt %) | | | | | | | | | | |

From the experimental results in Tables 1-2 to 1-5, it was demonstrated that the method for producing a styrene monomer of the present embodiment reduced deposits on the inner wall of the cooling tube or heat exchanger and reduced residue formation compared to the production method of the comparative examples. Therefore, it is considered that the blockage of piping caused by residues generated by thermal decomposition or gases produced during thermal decomposition can be suppressed, thereby preventing a reduction in the production volume of styrene per unit time.

1. A method for producing a styrene monomer, comprising:
a mixed liquid preparation step of preparing a mixed liquid by mixing a styrene-based resin composition containing a styrene-based polymer that includes styrene monomer units with a solvent;
a purification step of purifying the mixed liquid using a purifier;
a devolatilization step of devolatilizing the purified mixed liquid to obtain a fluid; and
a first thermal decomposition step of thermally decomposing the fluid to produce a first thermal decomposition liquid.

2. The method for producing a styrene monomer according to claim 1, further comprising a recovery step of recovering the styrene monomer by distilling the first thermal decomposition liquid.

3. The method for producing a styrene monomer according to claim 1, wherein the solvent is one or more selected from the group consisting of toluene, methyl ethyl ketone, and ethylbenzene.

4. The method for producing a styrene monomer according to claim 1, wherein the fluid contains 10 mass % or more and 100 mass % or less of the styrene-based polymer per 100 mass % of a total amount of the fluid.

5. The method for producing a styrene monomer according to claim 1, wherein the purified mixed liquid contains 5 to 100 mass % of the styrene-based polymer per 100 mass % of a total amount of the purified mixed liquid.

6. The method for producing a styrene monomer according to claim 1, wherein the mixed liquid contains 5 mass % or more of the styrene-based polymer per 100 mass % of the total amount of the mixed liquid.

7. The method for producing a styrene monomer according to claim 1, wherein an ambient temperature for mixing the styrene-based resin composition with the solvent in the mixed liquid preparation step is 0° C. or higher.

8. The method for producing a styrene monomer according to claim 1, wherein the devolatilization step is performed under reduced pressure.

9. The method for producing a styrene monomer according to claim 1, wherein the first thermal decomposition step is performed under reduced pressure.

10. The method for producing a styrene monomer according to claim 1, further comprising an analysis step of analyzing the first thermal decomposition liquid.

11. The method for producing a styrene monomer according to claim 1, wherein the first thermal decomposition step comprises cooling a thermally decomposed vapor containing the styrene monomer to obtain the first thermal decomposition liquid.

12. The method for producing a styrene monomer according to claim 1, wherein the styrene-based resin composition contains 20 mass % or less of impurities.

13. The method for producing a styrene monomer according to claim 1, further comprising a second thermal decomposition step of distilling the first thermal decomposition liquid to separate the first thermal decomposition liquid into a first fraction containing the styrene monomer and a second fraction having a lower styrene monomer concentration than that in the first fraction, then distilling the first fraction to separate the first fraction into a third fraction having a higher styrene monomer concentration than that in the first fraction and a fourth fraction having a lower styrene monomer concentration than that in the first fraction, and then thermally decomposing the fourth fraction again to produce a second thermal decomposition liquid.

14. The method for producing a styrene monomer according to claim 13, further comprising a recycling step of distilling the second thermal decomposition liquid to separate the second thermal decomposition liquid into a fifth fraction containing the styrene monomer and a sixth fraction having a lower styrene monomer concentration than that in the fifth fraction, and then recovering the styrene monomer from the fifth fraction.

15. The method for producing a styrene monomer according to claim 14, wherein the recycling step further comprises a step (I) of recovering the styrene monomer using the fifth fraction as a part of the first thermal decomposition liquid, and a step (II) of recovering the styrene monomer by distilling the fifth fraction separately from the first thermal decomposition liquid.

16. The method for producing a styrene monomer according to claim 1, wherein the first thermal decomposition step comprises heating the fluid to obtain the thermally decomposed vapor containing the styrene monomer and then cooling the thermally decomposed vapor to produce the first thermal decomposition liquid, wherein a concentration of a deposit-inducing substance contained in the first thermal decomposition liquid is less than 0.2 mass %.

17. The method for producing a styrene monomer according to claim 1, wherein the devolatilization step is a step of devolatilizing the mixed liquid to obtain the fluid using a flash drum, flash tank polymer heater, twin-screw devolatilizer, thin-film evaporator, or extruder.

18. The method for producing a styrene monomer according to claim 1, wherein the purifier employs one or more selected from the group consisting of filtration, decantation, centrifugal separation, centrifugal sedimentation, screw decanter, strainer, screen mesh, and filter.

19. The method for producing a styrene monomer according to claim 18, wherein the purifier is a purification mechanism combining centrifugal separation and filtration.
